SEQUENCE LISTING (15 sequences)

SEQ ID NO: 1 - DNA, 1671 bp, synthetic construct
  source 1..1671: mol_type = other DNA
atgagcgtgccggtgagtcagattccgagcctgaaagcgaaaggcgtgattatgcgccgcaacgcgaactatcatccgaacatttggggcgatcgctttattaactatgtgccggtggataaaatgaaccatacctgccatctgcaagcgattgaagaactgaaagatgcggtgcgccgcgaactgctgaccgcgaccggcctgagtcagctgaacctgattgatgcgattcagcgcctgggcgtgggctatcattttgaacgcgaactggaagaagcgctgcagcatgtgtatcataaaaaccattatcatgatgataccgaagataacctgtatagcattagcctgcgctttcgcctgctgcgtcagcatggctattatgtgagctgcgatattctgaacaaatttaaagatgaaaaagataactttaaagaaagcctgaccaccgatgtgccgggcatgctgagcctgtatgaagcggcgcatccgggcgtgcatggcgaagatattctggatgaagcgattgcgtttaccacgacccatctgaaaagcctggcgattgatcatctgcgcaacccgagcctggcgagccaagtgattcatgcgctgcgtcagccgctgcatcgcggcgtgccgcgcctggaaaaccgccgctatattagcatttatcaagatgaagtgagccataacaaagcgctggtgaaactgtttaaactggattttaacctggtgcagagcctgcataaaaaagaactgagcgaaattagccgctggtggaaagaactggatctggcgaacaaactgccgtttgcgcgcgatcgcctggtggaatgctatttttggattattggcgtgtattatgaaccgcagtatagcctggcgcgcaaaattctgaccaaaaccattgcgatgggcagcattattgatgatatttatgatgtgtatggcaccccggaagaactgaacctgtttaccgatgcgattgaacgctgggatgcgagctgcatggatcagctgccggaatatatgcagattttttatgaagcgctgctggatctgtataacgaaattgaaaaagaaattgcgaaagaaggctggagctatcgcgtgcattatgcgaaagaagcgatgaaaattctggcgcgcggctatcatgatgaaagcaaatggtttcataacaactatattccgaccatggaagaatatatgcatgtggcgctggtgacgagcggctataccatgctgaccacgagcagctttctgggcatggataacattgtgaccaaagaaacctttgattgggtgtttagcggcccgaaaattattcgcgcgagcggcaccattgcgcgcctgatggatgatgtgaaaagccataaatttgaacaagaacgcggccatgcggcgagcgcggtggagtgctacatggaacagcatggcgtgagcgaacaagaagtgtgcaaagaattttatcagcaagtgggcaacgcgtggaaagatattaaccaagattttctgaaaccgaccgatgtgccgatgaccattctgatgcgcgtgctgaacctggcgcgcgtgattgatgtggtgtataaagaaggcgatggctatacccatgtgggcaaagtgatgaaagaaaacgtggcgagcctgctgattgatccgattccggtgtaa

SEQ ID NO: 2 - DNA, 1731 bp, synthetic construct
  source 1..1731: mol_type = other DNA
atggcgacgagcgtgccaagcgtgctgttactgccggtgccgacctgcaccgatatgctggtgagcccggtggaaggcggcgatctgctgcattgcaaaccgcattttgatcatcatccgaacgtgtggggcgattattttctgacctttagcccgtgcaccccgagcatgctgctgaacatgaaacgcaaagcgcatgtgagcgaagaacaagtgcgccgcatgattctggaatgcagcagcggcccgaacctgcatgtgaaactggaactggtggataccctggaacgcctgtgcattgattatcattatgaaaaagaaattgaaaacgtgctgcgccgcgtgcatgaagaggaagatgataccgataaccattatgatctgcataccaccgcgctgcgcttttatctgctgcgcaaacatggctattatgcgagcccggatgtgtttcagcgctttcgcgatgaagaaggcaactttacccgcgatgataacaataacggcacccgcagcatgctgagcctgtataacgcggcgcatctgcgcattcatggcgaagaaattctggatgatgcgattgtgtttacccgcaactatctgcagagcgtggtgaaacatctgcagagcccgatggcggatgaagtgtgcagcgcgctgcgcaccccgctgtttcgccgcccgcgccgcgtggaagcgcgccattatattagcgtgtatgataaactgccgacccgcaacgaaaccattctggaatttgcgaaactggattttggcattctgcagagcctgtattgcgaagaactgaacattctgaccatgtggtggaaagaactgcagctgcaagatcatctgagctttgcgcgcgatcgcatggtggaaatgcatttttggatgctgggcgtgctgtttgaaccgcagtatagctatggccgcaccatgctgaccaaactgtttatttttgtgagcatttttgatgatatttatgataactatagcaccctggaagaaagcaaactgtttaccgaagcgattgaacgctgggatgaagaagcggcggaagaactgccgggctatatgaaatttttttataaaaaagtgctgaccaccatgaaaagcattgaaaccgatctgaaactgcaaggcaacaaacatgtggattatgtgaaaaacctgctgattgatgcgacccgctgcttttataacgaagtgaaatggcgcagcgaaggcgcggatcaagtggcggcgaccgtggaagaacatctgaaaattagcgtgccgagcagctgttgcatgcatgtgccggtgtatgcgtttgtggcgatgggcaacgatgtgaccaccgatgacgcgattaactggggcatggcgtatccgaaaattattacgagcagctgcattgtgggccgcctgctgaacgatattgcgagccatgaacgcgaacaaggcagcagtagcagtagcagcagtaccgttgaagcgtgcatgcgcgaacatggcggcattaccaaagaagaagcgtatgcgaaactgcgcgaactggtggaagaaagctggatggatatcgcgggcgaatgcctgcgcccggcggccgcgcaaccgccaccgttactggaagcggtggtgaacgcgacccgcgtgctggattttgtgtataaagatgatcaagatgcgtatacccatccgagcagcctgaaagataccattcatagcatttatattctgagcgtgtaa

SEQ ID NO: 3 - DNA, 969 bp, synthetic construct
  source 1..969: mol_type = other DNA
atggaaaaaattacctttccgaacctgtattgcccgtttccggaacgcaaaaatcagtattttgaagtgctgcaagattatgcgctgcagtgggtgctgcgctttaaactgattgatagcgaaagcctgtatcagcgctttagcaaagcgaaattttatctgctgaccgcgggcgcgtatccgcattgtcagctggaagaactgaagattgcgaacgatgttattagctggctgtttatttgggatgatcagtgcgatattagcgatctgggcaaaaaaccggaactgctgaaaatttggtgcaaccgctttctggaaattctgaacggcgcggaactgaccgcggatgatctgccgctgggctttgcgctgcgcgatattcgcaaccgcattattaaccgcggcagcattaccttttttcatcattttgtgcgcaactttgaagattatttttatggctgcattgaagaagcgcataaccgcgtgaccgtgagcattccggatgtggaagcgtatattaaaattcgcagcgcgaacgcggccgcggcgctgtgcctgaacctgattgaattttgcgatcgcgtgatgattccgtatagcctgcgcaaccatgataccctgaacaaactgacgcagatgaccattaacattctggcgtggagcaacgatatttttagcgcgccgcgcgaaattgcgaacggcgaagtgcataacctggtgtttgtgattcatcaccatcagaaaattccgctggaaaaagcgatgctggccgccgcggccatgcataaccaagaagtggaaaacctggtgaaactggaaagtcagattacctattttagcgcggaaattgatgcggaaattaccaaatatattagcggcctgcatgcgtggattcgcggcaacctggattggtatgcgcatagcggccgctatcagattaccgaaaaactggaactgatggcgagctaa

SEQ ID NO: 4 - DNA, 10478 bp, synthetic construct
  source 1..10478: mol_type = other DNA
ggggaattgtgagcggataacaattcccctgtagaaataattttgtttaactttaataaggagatataccatgggcagcagccatcaccatcatcaccacagccaggatccgatgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctacgatgcgcctttttctagtatgatgtacgatgggttaacggatgcctttagtggtcaagcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtgtcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtattggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagtgattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaaatttttcaacaagcagagttaagttatccatctatcgtaaaacggggcggcggcttaagagatttgcagtatcgtgcttttgatgaatcatttatatctgtcgactttttagtagatgttaaggatgcaatgggggcaaatatcgttaacgctatgttggaaggtgtggtcgagttgttccgtgaatggtttgcggagcaaaaaattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgactagttggacgctggatggcgaacaactaattggtgaaatttcagttccgcttgctttagccacggttggcggtgccacaaaagtcttacctaaatctcaagcagccgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaagggggaggtggctccgggggaggtggctccatgacaattgggattgataaaattagtttttttgtgcccccttattatattgatatgacggcactggctgaagccagaaatgtagaccctggaaaatttcatattggtattgggcaagaccaaatggcggtgaacccaatcagccaagatattgtgacatttgcagccaatgccgcagaagcgatcttgaccaaagaagataaagaggccattgatatggtgattgtcgggactgagtccagtatcgatgagtcaaaagcggccgcagttgtcttacatcgtttaatggggattcaacctttcgctcgctctttcgaaatcaaggaagcttgttacggagcaacagcaggcttacagttagctaagaatcacgtagccttacatccagataaaaaagtcttggttgtagcagcagatattgcaaaatatggattaaattctggcggtgagcctacacaaggagctggggcggttgcaatgttagttgctagtgaaccgcgcatcttggctttaaaagaggataatgtgatgctgacgcaagatatctatgacttttggcgtccaacaggccatccgtatcctatggtcgatggtcctttgtcaaacgaaacctacatccaatcttttgcccaagtctgggatgaacataaaaaaagaaccggtcttgattttgcagattatgatgctttagcgttccatattccttacacaaaaatgggcaaaaaagccttattagcaaaaatctccgaccaaactgaagcagaacaggaacgaattttagcccgttatgaagaaagcatcatctatagtcgtcgcgtaggaaacttgtatacgggttcactttatctgggactcatttcccttttagaaaatgcaacgactttaaccgcaggcaatcaaattgggttattcagttatggttctggtgctgtcgctgaatttttcactggtgaattagtagctggttatcaaaatcatttacaaaaagaaactcatttagcactgctagataatcggacagaactttctatcgctgaatatgaagccatgtttgcagaaactttagacacagatattgatcaaacgttagaagatgaattaaaatatagtatttctgctattaataataccgttcgctcttatcgaaactaactgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctagtgctcaaattcagcaaaattgaaaaactgttgaggaataatatggtttcatgttctgcgcccgggaaaatctatcttttcggagaacatgcggttgtttacggagaaaccgcaatagcgtgtgcagtagagttaaggacccgggtgcgggcggagttaaatgactccataactatccagtctcagatcggcaggacaggtcttgattttgaaaaacatccctatgtctctgcagtgattgaaaaaatgagaaaatctatccccataaatggtgtttttttaactgttgattccgacattcctgttgggtcagggctcggctcatctgctgctgttacgattgcaagcataggagctctcaacgaacttttcggattcgggctttcgcttcaggaaattgcgaaactggggcatgaaattgagataaaagttcagggtgcagcgagccctactgacacctatgtttctactttcggaggagtcgttaccatccctgaaaggagaaagcttaagactcctgactgtggaattgttataggggacaccggagttttttcttctacaaaagagcttgtggcaaacgtcaggcagctccgcgaaagttaccctgatcttatcgaacctcttatgacttctattggcaaaatctccagaatcggtgagcaacttgtactttccggggactatgcttctattggcaggcttatgaatgtaaatcagggactgcttgatgcacttggagttaatatccttgagctttcacagcttatctattctgcaagggcagcaggagctttcggggcaaaaattactggagcgggaggcggtggttgtatggttgcgctaactgcaccggagaaatgtaatcaggtagcggaagccattgcaggtgcggggggcaaagtgaccattacaaaacccacggaacaggggttgaaggtcgattgaggaggcagatcaaatgattgctgttaaaacttgcggaaaactctatttggcaggtgaatatgctattttagagccagggcagttagctttgataaaggatattcccatctatatgagggctgagattgctttttctgatagctaccgtatctattcagatatgtttgatttcgcagtggacttaaggcccaatcctgactatagcttgattcaagaaacgattgctttgatgggagacttcctcgctgttcgtggtcagaatttaagacctttttctctagaaatctgtggcaaaatggaacgagaagggaaaaagtttggtctaggttctagtggcagcgtcgttgtcttggttgtcaaggctttactggctctctataatctttcggttgatcagaatctcttgttcaagctgactagcgctgtcttgctcaagcgaggagacaatggttccatgggcgaccttgcctgtattgtggcagaggatttggttctctaccagtcatttgatcgccagaaggtggctgcttggttagaagaagaaaacttggcgacagttctggagcgtgattggggattttttatctcacaagtgaaaccaactttagaatgtgatttcttagtgggatggaccaaggaagtggctgtatcgagtcacatggtccagcaaatcaagcaaaatatcaatcaaaattttttaacttcctcaaaagaaacggtggtttctttggtcgaagccttggagcaggggaaagccgaaaaagttatcgagcaagtagaagtagccagcaagcttttagaaggcttgagtacagatgtttacacgcctttgcttagacagttgaaagaagccagtcaagatttgcagaccgttgccaagagtagtggtgctggtggtggtgactgtggcatcgccctgagttttgatgcgcaatcaaccgaaaccttaaaaaatcgttgggccgatctggggattgagctcttatatcaagaaaggataggacatgacgacaaatcgtaaataggaggtaatactcatggatagagagcctgtaacagtacgttcctacgcaaatattgctattatcaaatattggggaaagaaaaaagaaaaagagatggtgcctgctactagcagtatttctctaactttggaaaatatgtatacagagacgaccttgtcgcctttaccagccaatgtaacagctgacgaattttacatcaatggtcagctacaaaatgaggtcgagcatgctaagatgagtaagattattgaccgttatcgtccagctggtgagggctttgtccgtatcgatactcaaaacaatatgcctactgcagcgggcctgtcctcaagttctagtggtttgtccgccctggtcaaggcttgtaatgcttatttcaagcttggattggatagaagtcagttggcacaggaagccaaatttgcctcaggctcttcttctcggagtttttatggaccactaggagcctgggataaggatagtggagaaatttaccctgtagagacagacttgaaactagctatgattatgttggtgctagaggacaagaaaaaaccaatctctagccgtgacgggatgaaactttgtgtggaaacctcgacgacttttgacgactgggttcgtcagtctgagaaggactatcaggatatgctgatttatctcaaggaaaatgattttgccaagattggagaattaacggagaaaaatgccctggctatgcatgctacgacaaagactgctagtccagccttttcttatctgacggatgcctcttatgaggctatggactttgtccgccagcttcgtgagaaaggagaggcctgctactttaccatggatgctggtcccaatgttaaggtcttctgtcaggagaaagacttggagcatttatcagaaattttcggtcagcgttatcgcttgattgtgtcaaaaacaaaggatttgagtcaagatgattgctgttaactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 5 - DNA, 1719 bp, synthetic construct
  source 1..1719: mol_type = other DNA
atgaaacaaacggttgcagcttatatcgccaaaacactcgaatcggcaggggtgaaacgcatctggggagtcacaggcgactctctgaacggtcttagtgacagtcttaatcgcatgggcaccatcgagtggatgtccacccgccacgaagaagtggcggcctttgccgctggcgctgaagcacaacttagcggagaactggcggtctgtgccggatcgtgcggccccggcaacctgcacttaatcaacggcctgttcgattgccaccgcaatcacgttccggtactggcgattgccgctcatattccctccagcgaaattggcagcggctatttccaggaaacccacccacaagagctattccgcgaatgtagtcactattgcgagctggtttccagcccggagcagatcccacaagtactggcgattgccatgcgcaaagcggtgcttaaccgtggcgtttcgattgtcgtgttaccaggcgacgtggcgttaaaacctgcgccagaaggggcaaccatgcactggtatcatgcgccacaaccagtcgtgacgccggaagaagaagagttacgcaaactggcgcaactgctgcgttattccagcaatatcgccctgatgtgtggcagcggctgcgcgggggcgcataaagagttagttgagtttgccgggaaaattaaagcgcctattgttcatgccctgcgcggtaaagaacatgtcgaatacgataatccgtatgatgttggaatgaccgggttaatcggcttctcgtcaggtttccataccatgatgaacgccgacacgttagtgctactcggcacgcaatttccctaccgcgccttctacccgaccgatgccaaaatcattcagattgatatcaacccagccagcatcggcgctcacagcaaggtggatatggcactggtcggcgatatcaagtcgactctgcgtgcattgcttccattggtggaagaaaaagccgatcgcaagtttctggataaagcgctggaagattaccgcgacgcccgcaaagggctggacgatttagctaaaccgagcgagaaagccattcacccgcaatatctggcgcagcaaattagtcattttgccgccgatgacgctattttcacctgtgacgttggtacgccaacggtgtgggcggcacgttatctaaaaatgaacggcaagcgtcgcctgttaggttcgtttaaccacggttcgatggctaacgccatgccgcaggcgctgggtgcgcaggcgacagagccagaacgtcaggtggtcgccatgtgcggcgatggcggttttagcatgttgatgggcgatttcctctcagtagtgcagatgaaactgccagtgaaaattgtcgtctttaacaacagcgtgctgggctttgtggcgatggagatgaaagctggtggctatttgactgacggcaccgaactacacgacacaaactttgcccgcattgccgaagcgtgcggcattacgggtatccgtgtagaaaaagcgtctgaagttgatgaagccctgcaacgcgccttctccatcgacggtccggtgctggtggatgtggtggtcgccaaagaagagttagccattccaccgcagatcaaactcgaacaggccaaaggatttagcttgtatatgttgcgcgcaatcatcagcggacgcggcgatgaagtgatcgaactggcgaaaacgaactggctaaggtaa

SEQ ID NO: 6 - DNA, 2379 bp, synthetic construct
  source 1..2379: mol_type = other DNA
atgtccaacaatggctcgtcaccgctggtgctttggtataaccaactcggcatgaatgatgtagacagggttgggggcaaaaatgcctccctgggtgaaatgattactaacctttccggaatgggtgtttccgttccgaatggtttcgccacaaccgccgacgcgtttaaccagtttctggaccaaagcggcgtaaaccagcgcatttatgaactgctggataaaacggatattgacgatgttactcagcttgcgaaagcgggcgcgcaaatccgccagtggattatcgacactcccttccagcctgagctggaaaacgccatcagcgaagcctatgcacagctttctgccgatgacgaaaacgcctcttttgcggtgcgctcctccgccaccgcagaagatatgccggacgcttcttttgccggtcagcaggaaaccttcctcaacgttcagggttttgacgccgttctcgtggcagtgaaacatgtatttgcttctctgtttaacgatcgcgccatctcttatcgtgtgcaccagggttacgatcaccgtggtgtggcgctctccgccggtgttcaacggatggtgcgctctgacctcgcatcatctggcgtgatgttctccattgataccgaatccggctttgaccaggtggtgtttatcacttccgcatggggccttggtgagatggtcgtgcagggtgcggttaacccggatgagttttacgtgcataaaccgacactggcggcgaatcgcccggctatcgtgcgccgcaccatggggtcgaaaaaaatccgcatggtttacgcgccgacccaggagcacggcaagcaggttaaaatcgaagacgtaccgcaggaacagcgtgacatcttctcgctgaccaacgaagaagtgcaggaactggcaaaacaggccgtacaaattgagaaacactacggtcgcccgatggatattgagtgggcgaaagatggccacaccggtaaactgttcattgtgcaggcgcgtccggaaaccgtgcgctcacgcggtcaggtcatggagcgttatacgctgcattcacagggtaagattatcgccgaaggccgtgctatcggtcatcgcatcggtgcgggtccggtgaaagtcatccatgatatcagcgaaatgaaccgcatcgaacctggtgacgtgctggtcactgacatgaccgacccggactgggaaccgatcatgaagaaagcatctgccatcgtcaccaaccgtggcggtcgtacctgtcacgcggcgatcatcgctcgtgaactgggcattccggcggtagtgggctgtggtgatgcaacagaacggatgaaagacggtgagaacgtcactgtttcttgtgccgaaggtgataccggttacgtctatgcggagttgctggaatttagcgtgaaaagctccagcgtagaaacgatgccggatctgccgttgaaagtgatgatgaacgtcggtaacccggaccgagctttcgacttcgcctgtctgccgaacgaaggcgtgggacttgcgcgtctggaatttatcatcaaccgtatgattggcgtccacccacgcgcactgcttgagtttgacgatcaggaaccgcagttgcaaaacgaaatccgcgagatgatgaaaggttttgattctccgcgtgaattttacgttggtcgtctgactgaagggatcgcgacgctgggtgccgcgttttatccgaagcgcgtcattgtccgtctctctgattttaaatcgaacgaatatgccaacctggtcggtggtgagcgttacgagccagatgaagagaacccgatgctcggcttccgtggcgcgggacgctatatttccgacagcttccgcgactgtttcgcgctggagtgcgaagcagtgaaacgtgtgcgcaacgacatggggctgaccaacgttgagatcatgatcccgttcgtgcgaaccgtagatcaggcgaaagcggtggttgaggaactggcgcgtcaggggctgaaacgtggtgagaacgggctgaaaatcatcatgatgtgtgaaatcccgtccaacgccttgctggccgagcagttcctcgaatatttcgacggcttctcaattggctcaaacgacatgacgcagctggcgctcggtctggatcgtgactccggcgtggtgtctgaactgttcgatgagcgcaacgatgcggtgaaagcactgctgtcgatggcgattcgtgccgcgaagaaacagggcaaatatgtcgggatttgcggtcagggtccgtccgaccacgaagactttgccgcatggttgatggaagaggggatcgatagcctgtctctgaacccggacaccgtggtgcaaacctggttaagcctggctgaactgaagaaataa

SEQ ID NO: 7 - DNA, 1434 bp, synthetic construct
  source 1..1434: mol_type = other DNA
atgtttaagaatgcatttgctaacctgcaaaaggtcggtaaatcgctgatgctgccggtatccgtactgcctatcgcaggtattctgctgggcgtcggttccgcgaatttcagctggctgcccgccgttgtatcgcatgttatggcagaagcaggcggttccgtctttgcaaacatgccactgatttttgcgatcggtgtcgccctcggctttaccaataacgatggcgtatccgcgctggccgcagttgttgcctatggcatcatggttaaaaccatggccgtggttgcgccactggtactgcatttacctgctgaagaaatcgcctctaaacacctggcggatactggcgtactcggagggattatctccggtgcgatcgcagcgtacatgtttaaccgtttctaccgtattaagctgcctgagtatcttggcttctttgccggtaaacgctttgtgccgatcatttctggcctggctgccatctttactggcgttgtgctgtccttcatttggccgccgattggttctgcaatccagaccttctctcagtgggctgcttaccagaacccggtagttgcgtttggcatttacggtttcatcgaacgttgcctggtaccgtttggtctgcaccacatctggaacgtacctttccagatgcagattggtgaatacaccaacgcagcaggtcaggttttccacggcgacattccgcgttatatggcgggtgacccgactgcgggtaaactgtctggtggcttcctgttcaaaatgtacggtctgccagctgccgcaattgctatctggcactctgctaaaccagaaaaccgcgcgaaagtgggcggtattatgatctccgcggcgctgacctcgttcctgaccggtatcaccgagccgatcgagttctccttcatgttcgttgcgccgatcctgtacatcatccacgcgattctggcaggcctggcattcccaatctgtattcttctggggatgcgtgacggtacgtcgttctcgcacggtctgatcgacttcatcgttctgtctggtaacagcagcaaactgtggctgttcccgatcgtcggtatcggttatgcgattgtttactacaccatcttccgcgtgctgattaaagcactggatctgaaaacgccgggtcgtgaagacgcgactgaagatgcaaaagcgacaggtaccagcgaaatggcaccggctctggttgctgcatttggtggtaaagaaaacattactaacctcgacgcatgtattacccgtctgcgcgtcagcgttgctgatgtgtctaaagtggatcaggccggcctgaagaaactgggcgcagcgggcgtagtggttgctggttctggtgttcaggcgattttcggtactaaatccgacaacctgaaaaccgagatggatgagtacatccgtaaccactaa

SEQ ID NO: 8 - DNA, 510 bp, synthetic construct
  source 1..510: mol_type = other DNA
atgggtttgttcgataaactgaaatctctggtttccgacgacaagaaggataccggaactattgagatcattgctccgctctctggcgagatcgtcaatatcgaagacgtgccggatgtcgtttttgcggaaaaaatcgttggtgatggtattgctatcaaaccaacgggtaacaaaatggtcgcgccagtagacggcaccattggtaaaatctttgaaaccaaccacgcattctctatcgaatctgatagcggcgttgaactgttcgtccacttcggtatcgacaccgttgaactgaaaggcgaaggcttcaagcgtattgctgaagaaggtcagcgcgtgaaagttggcgatactgtcattgaatttgatctgccgctgctggaagagaaagccaagtctaccctgactccggttgttatctccaacatggacgaaatcaaagaactgatcaaactgtccggtagcgtaaccgtgggtgaaaccccggttatccgcatcaagaagtaa

SEQ ID NO: 9 - DNA, 2676 bp, synthetic construct
  source 1..2676: mol_type = other DNA
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagatcctgctggatacctactacggtcgtgattatgtagaaggtgaaactgcagcgaaaaaagaagccgctccggctaaagctgagaaaaaagcgaaaaaatccgcttaa

SEQ ID NO: 10 - DNA, 990 bp, synthetic construct
  source 1..990: mol_type = other DNA
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgacgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactaccatctgttgaacgaagccgccttcgatcagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctgttctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggtttaa

SEQ ID NO: 11 - DNA, 1476 bp, synthetic construct
  source 1..1476: mol_type = other DNA
atggcggtaacgcaaacagcccaggcctgtgacctggtcattttcggcgcgaaaggcgaccttgcgcgtcgtaaattgctgccttccctgtatcaactggaaaaagccggtcagctcaacccggacacccggattatcggcgtagggcgtgctgactgggataaagcggcgtataccaaagttgtccgcgaggcgctcgaaactttcatgaaagaaaccattgatgaaggtttatgggacaccctgagtgcacgtctggatttttgtaatctcgatgtcaatgacactgctgcattcaaccgtctcggcgcgatgctggatcaaaaaaatcgtatcaccattaactactttgccatgccgcccagcacttttggcgcaatttgcaaagggcttggcgaggcaaaactgaatgctaaaccggcacgcgtagtcatggagaaaccgctggggacgtcgctggcgacctcgcaggaaatcaatgatcaggttggcgaatacttcgaggagtgccaggtttaccgtatcgaccactatcttggtaaagaaacggtgctgaacctgttggcgctgcgttttgctaactccctgtttgtgaataactgggacaatcgcaccattgatcatgttgagattaccgtggcagaagaagtggggatcgaagggcgctggggctattttgataaagccggtcagatgcgcgatatgatccaaaaccacctgctgcaaattctctgcatgattgcgatgtctccgccatctgacctgagcgcagacagcatccgcgatgaaaaagtgaaagtactgaagtccctgcgccgcatcgaccgctccaacgtacgcgaaaaaaccgtacgtgggcaatatactgcgggcttcgcccagggcaaaaaagtgccgggatatctggaagaagagggcgcgaacaagagcagcaatacagaaaccttcgtggcgatccgcgtcgacattgataactggcgctgggccggtgtgccattctacctgcgtactggtaaacgtctgccgaccaaatgttctgaagtcgtggtctatttcaaaacacctgaactgaatctgtttaaagagtcgtggcaggatctgccgcagaataaactgactatccgtctgcaacctgatgaaggcgtggatatccaggtactgaataaagttcctggccttgaccacaaacataacctgcaaatcaccaagctggatctgagctattcagaaacctttaatcagacgcatctggcggatgcctatgaacgtctgctgctggaaaccatgcgtggtattcaggcactgtttgttcgtcgcgacgaagtggaagaagcctggaaatgggtagactccattactgaggcgtgggcgatggacaatgatgcgccgaaaccgtatcaggccggaacctggggacccgttgcctcggtggcgatgattacccgtgatggtcgttcctggaatgagtttgagtaa

SEQ ID NO: 12 - DNA, 1863 bp, synthetic construct
  source 1..1863: mol_type = other DNA
atgagttttgatattgccaaatacccgaccctggcactggtcgactccacccaggagttacgactgttgccgaaagagagtttaccgaaactctgcgacgaactgcgccgctatttactcgacagcgtgagccgttccagcgggcacttcgcctccgggctgggcacggtcgaactgaccgtggcgctgcactatgtctacaacaccccgtttgaccaattgatttgggatgtggggcatcaggcttatccgcataaaattttgaccggacgccgcgacaaaatcggcaccatccgtcagaaaggcggtctgcacccgttcccgtggcgcggcgaaagcgaatatgacgtattaagcgtcgggcattcatcaacctccatcagtgccggaattggtattgcggttgctgccgagaaagaaggcaaaaatcgccgcaccgtctgtgtaatcggcgatggtgcgattaccgcaggcatggcgtttgaagcgatgaatcacgcgggcgatatccgtcctgatatgctggtggttctcaacgacaatgaaatgtcgatttccgaaaatgtcggcgcgctcaacaaccatctggcgcagctgctttccggtaagctttactcttcactgcgcgaaggcgggaaaaaagttttctctggcgttccgccaattaaagagctgctcaaacgcaccgaagaacatattaaaggcatggtagtgcctggcacgttgtttgaagagctgggctttaactacatcggcccggttgacggtcacgatgtgctggggcttatcaccacgcttaagaacatgcgcgacctgaaaggcccgcagttcctgcatatcatgaccaaaaaaggtcgtggttatgaaccggcagaaaaagacccaatcactttccacgccgtgcctaaatttgatccctccagcggttgtttgccgaaaagtagcggcggtttgccgagctattcaaaaatctttggcgactggttgtgtgaaacggcagcgaaagacaacaagctgatggcgattactccggcaatgcgtgaaggttccggcatggtcgagttttcacgtaaattcccggatcgctacttcgacgtggcaattgccgagcaacacgcagtgacctttgccgccggtctggcgattggtgggtacaaacccattgtcgcgatttactccactttcctgcaacgcgcctatgatcaggtgctgcatgacgtggcgattcaaaagctaccggtcctgttcgccattgaccgcgcgggcattgttggtgctgacggtcaaactcaccagggcgcttttgatctctcttacctgcgctgcataccagaaatggtcattatgaccccgagcgatgaaaacgaatgtcgccagatgctctataccggctatcactataacgatggcccgtccgcggtgcgctacccgcgcggcaacgcggttggcgtggaactgacgccgctggaaaaactaccaattggcaaaggcattgtgaagcgtcgtggcgagaaactggcgatccttaactttggtacgctgatgccagaagcggcgaaagtcgccgaatcgctgaacgccacgctggtcgatatgcgttttgtgaaaccgcttgatgaagcgttaattctggaaatggccaccagccatgaagcgctggtcaccgtagaagaaaacgccattatgggcggcgcaggtagcggagtgaacgaagtgctgatggcccatcgtaaaccagtacccgtgctgaacattggtctgccggacttctttattccgcaaggaactcaggaagaaatgcgcgccgaactcggcctcgatgccgccggtatggaagccaaaatcaaggcctggctggcataa

SEQ ID NO: 13 - DNA, 1119 bp, synthetic construct
  source 1..1119: mol_type = other DNA
atgcataaccaggctccaattcaacgtagaaaatcaacacgtatttacgttgggaatgtgccgattggcgatggtgctcccatcgccgtacagtccatgaccaatacgcgtacgacagacgtcgaagcaacggtcaatcaaatcaaggcgctggaacgcgttggcgctgatatcgtccgtgtatccgtaccgacgatggacgcggcagaagcgttcaaactcatcaaacagcaggttaacgtgccgctggtggctgacatccacttcgactatcgcattgcgctgaaagtagcggaatacggcgtcgattgtctgcgtattaaccctggcaatatcggtaatgaagagcgtattcgcatggtggttgactgtgcgcgcgataaaaacattccgatccgtattggcgttaacgccggatcgctggaaaaagatctgcaagaaaagtatggcgaaccgacgccgcaggcgttgctggaatctgccatgcgtcatgttgatcatctcgatcgcctgaacttcgatcagttcaaagtcagcgtgaaagcgtctgacgtcttcctcgctgttgagtcttatcgtttgctggcaaaacagatcgatcagccgttgcatctggggatcaccgaagccggtggtgcgcgcagcggggcagtaaaatccgccattggtttaggtctgctgctgtctgaaggcatcggcgacacgctgcgcgtatcgctggcggccgatccggtcgaagagatcaaagtcggtttcgatattttgaaatcgctgcgtatccgttcgcgagggatcaacttcatcgcctgcccgacctgttcgcgtcaggaatttgatgttatcggtacggttaacgcgctggagcaacgcctggaagatatcatcactccgatggacgtttcgattatcggctgcgtggtgaatggcccaggtgaggcgctggtttctacactcggcgtcaccggcggcaacaagaaaagcggcctctatgaagatggcgtgcgcaaagaccgtctggacaacaacgatatgatcgaccagctggaagcacgcattcgtgcgaaagccagtcagctggacgaagcgcgtcgaattgacgttcagcaggttgaaaaataa

SEQ ID NO: 14 - DNA, 10234 bp, synthetic construct
  source 1..10234: mol_type = other DNA
ggggaattgtgagcggataacaattcccctgtagaaataattttgtttaactttaataaggagatataccatgggcatggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccaccggtcatatgtttggcgttagcacaaacacgctggacgcacccgctgctgccgtagagtgtatccacgcttactcattaattcatgatgatttaccggcgatggatgatgacgatctgcgccgcggtttgccgacctgccatgtgaagtttggcgaagcaaacgcgattctcgctggcgacgctttacaaacgctggcgttctcgattctaagcgatgccgatatgccggaagtgtcggatcgcgacagaatttcgatgatttctgaactggcgagcgccagcggtattgccggaatgtgcggtggtcaggcactagatttagacgcggaaggcaaacacgtacctctggacgcgcttgagcgtattcatcgtcataaaaccggcgcattgattcgcgccgccgttcgccttggtgcattaagcgccggagataaagggcgtcgtgctctgccagtactcgacaagtacgcagagagcatcggccttgccttccaggttcaagatgacatcctggatgtggtaggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataatcgttgcaaccgacttcaaggagacttttatatggaaaaaattacctttccgaacctgtattgcccgtttccggaacgcaaaaatcagtattttgaagtgctgcaagattatgcgctgcagtgggtgctgcgctttaaactgattgatagcgaaagcctgtatcagcgctttagcaaagcgaaattttatctgctgaccgcgggcgcgtatccgcattgtcagctggaagaactgaagattgcgaacgatgttattagctggctgtttatttgggatgatcagtgcgatattagcgatctgggcaaaaaaccggaactgctgaaaatttggtgcaaccgctttctggaaattctgaacggcgcggaactgaccgcggatgatctgccgctgggctttgcgctgcgcgatattcgcaaccgcattattaaccgcggcagcattaccttttttcatcattttgtgcgcaactttgaagattatttttatggctgcattgaagaagcgcataaccgcgtgaccgtgagcattccggatgtggaagcgtatattaaaattcgcagcgcgaacgcggccgcggcgctgtgcctgaacctgattgaattttgcgatcgcgtgatgattccgtatagcctgcgcaaccatgataccctgaacaaactgacgcagatgaccattaacattctggcgtggagcaacgatatttttagcgcgccgcgcgaaattgcgaacggcgaagtgcataacctggtgtttgtgattcatcaccatcagaaaattccgctggaaaaagcgatgctggccgccgcggccatgcataaccaagaagtggaaaacctggtgaaactggaaagtcagattacctattttagcgcggaaattgatgcggaaattaccaaatatattagcggcctgcatgcgtggattcgcggcaacctggattggagcgcgcataacggccgctatcagattaccgaaaaactggaactgatggcgagctaaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcatgacgacaaatcgtaaggacgagcatatcctctatgcccttgagcagaaaagttcctataatagctttgatgaggtggagctgattcattcttccttgcctctttacaatctggatgaaatcgatctttcgacagagtttgctggtcggaagtgggactttcctttttatatcaatgccatgactggtggaagtaataagggaagagaaatcaatcaaaagctggctcaggtggcggaatcctgtggtattttatttgtaacgggttcttatagcgcagccctcaaaaatccaacggatgattctttttctgtcaagtctagtcatcccaatctcctccttggaaccaatattggattggacaagcctgtcgagttaggacttcagactgtagaagagatgaatcctgttctattgcaagtgcatgtcaatgtcatgcaggaattactcatgcccgagggagaaaggaagtttagaagctggcaatcgcatctagcagattatagcaagcaaattcccgttcctattgtcctcaaggaagtgggctttggaatggatgccaagacaatcgaaagagcctatgaattcggtgttcgtacagtggacctatcgggtcgtggtggcaccagctttgcctatatcgaaaaccgtcgtagtggccagcgtgattacctcaatcaatggggtcagtctaccatgcaggcccttctcaatgcccaagaatggaaagataaggtcgaactcttggttagtggaggggttcggaatccgctggatatgattaagtgcttggtttttggtgctaaggctgtgggattgtcacgaaccgttctggaattggttgaaacctacacagttgaagaagtgattggcattgttcaaggctggaaagcagatctacgcttgattatgtgttcccttaactgtgccaccatagcagatctacaaaaagtagactatcttctttatggaaaattaaaagaagcaaatgatcagatgaaaaaggcgtaaacattacgtaagtaaggggggctaggatgtcaaaagtaatgaagacaatggatggaaacaccgcggcggcgcacgtagcctacgctttcaccgaagtcgcggcgatctatccgatcaccccatcgtcgccgatggctgaacacgtggacgagtggagcgcccatggtcgtaaaaatctgttcggccaagaagtgaaggtgattgaaatgcagagcgaagcaggtgctgcgggtgcggtgcacggctctctagcggcgggtgcattgactacaacctttaccgcatcccaaggtctgttgttaatgatcccgaatatgtataaaatcgctggcgagctgctgccgggtgttttccacgtgagcgcgcgtgcgctggcgtcgcacgcactgtcaatcttcggcgatcatcaggacgtcatggcgtgccgtcagaccggtttcgcgttgctggcgtccggctctgtccaggaggtgatggatctgggtagcgttgcacacctggccgcgatcaagggtcgtgttccgttcctgcatttttttgatggttttcgcaccagccacgaataccagaaaatcgaggtgatggactacgaagatctgcgtaaactgctggatatggacgccgttcgcgaattcaaaaagcgcgctttgaacccggagcacccggttacgcgtggtacagcgcagaacccggacatctacttccaggagcgcgaggcgtcgaatcgttactataatgccgtgccggagattgtcgaagagtacatgaaagaaattagcaagatcaccggtcgcgaatacaaactgtttaattattacggcgctccggatgcggagcgcatcgtgatcgcaatgggctctgttaccgaaaccatcgaggagactatcgattacctccttaagaaaggcgagaaagttggtgttgttaaggtgcacttgtatcgaccgttcagctttaaacacttcatggatgccattcccaagacggttaagaagattgccgttttggaccgtaccaaagaagctggtgcgttcggcgagccgctgtatgaagatgtgagagccgctttctatgatagcgaaatgaagccgattattgtcggcggacgttacggcctgggctctaaggacaccaccccagcgcagatcgtggcggtattcgacaacctgaaatcggataccccgaaaaacaactttaccattggcattgttgatgatgtcacgtacacctcactgccggtcggcgaggagattgagacgaccgcggagggcaccatttcttgtaaattttggggtttcggtagcgatggcactgtgggcgcgaataaatcggctatccaaattatcggtgataacacggacatgtacgcacaggcgtactttagctacgacagcaaaaagtccggtggtgttaccattagccaccttcgttttggtaaaaagccgatccgtagcacctacttaatcaacaacgcagacttcgtggcgtgccataaacaagcatacgtgtacaactatgacgtgctggctggtctgaaaaaaggcggcaccttcctgttgaactgcacctggaaaccggaggagctggacgagaagttgccggcgagcatgaaacgttatattgcgaaaaacaacattaacttctatattattaacgcagttgatatcgccaaagagctcggtctcggggcgcgtatcaatatgattatgcagagcgcgttcttcaaattggcgaacatcatcccgatcgatgaagccgtgaagcacttgaaggacgctattgtgaagagctatggtcataaaggagaaaagattgtgaacatgaactacgcagcggtggatcgcggtatcgatgcgctggttaaggttgacgtgccggcttcctgggcaaacgcagaggacgaagcgaaagtggaacgtaatgttccggatttcattaagaacatcgcagatgttatgaatcgtcaagagggcgataaactgccggttagcgcgtttgtaggtatggaagacggcacgtttccgatgggcactgccgcgtacgaaaagcgcggtatcgcggtggatgttccggaatggcagatcgacaattgcatccaatgcaaccagtgcgcatacgtttgtccgcatgctgcgatccgcccattcctgttgaatgaggaggaagtgaaaaacgccccagagggcttcacgagcaagaaggcgatcggcaagggtctggaaggtcttaacttccgtattcaagtttccgtcttagactgtaccggctgcggcgtgtgcgctaacacctgtccgtccaaggagaaatcgctgataatgaaaccgttggagacgcagctggaccaggcgaaaaattgggaatacgccatgagcctgagctataaagagaatccgctgggtaccgataccgtgaagggtagccaatttgaaaagccgctgttagaattttccggcgcttgcgctggctgcggtgaaactccgtatgcgcgtttggttacccagctgtttggggaccgcatgctgattgctaatgcgaccggctgcagctccatatggggtggctctgcaccaagcaccccgtataccgttaataaagacggtcatggtccggcatgggcgaatagcctgttcgaagacaacgccgagttcggctttggtatggcgctagctgtgaaacagcagcgtgaaaagttggcggacatcgtcaaagaggccctggagttggacctgacccaagacctcaagaacgcgttgaagctgtggctggataatttcaacagctccgaaatcaccaaaaagactgcgaacatcatcgtttcgctgattcaagactataagaccgacgactccaaggtgaaagaattgctcaacgagattctggaccgtaaagagtatctggtgaagaaatcacaatggatatttggtggcgacggctgggcatacgacatcggcttcggcggcctggaccacgtgctggcgtctggtgaagatgttaacgttctggtctttgataccgaagtctatagcaatacgggcggacaaagcagtaaagcgaccccggttggtgccattgcgcagtttgctgcggctggtaagggcatcggcaagaaggatttgggacgtattgcgatgagctacggttatgtatacgttgcccaaatcgcaatgggtgctaaccaagcgcaaaccatcaaggcattgaaagaggccgagagctacccgggtccatctctaattatcgcatacgcaccgtgtattaatcatggtatcaaactgggtatgggttgcagccagatcgaagaaaagaaagcagtcgaggctggctattggcatctctaccgctataatccgatgctgaaggccgaaggcaagaacccttttattctggacagcaaggcaccgaccgcgtcctacaaagagttcattatgggtgaggtgcgttactccagtctggcgaagacgtttccggagcgcgccgaggcactttttgaaaaagcggaggagttagctaaagaaaagtatgagacatacaagaaactggctgagcagaattaactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgcagcgctcttccgcttcctcgctcactgactcgctacgctcggtcgttcgactgcggcgagcggtgtcagctcactcaaaagcggtaatacggttatccacagaatcaggggataaagccggaaagaacatgtgagcaaaaagcaaagcaccggaagaagccaacgccgcaggcgtttttccataggctccgcccccctgacgagcatcacaaaaatcgacgctcaagccagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgttggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccattggtaactgatttagaggactttgtcttgaagttatgcacctgttaaggctaaactgaaagaacagattttggtgagtgcggtcctccaacccacttaccttggttcaaagagttggtagctcagcgaaccttgagaaaaccaccgttggtagcggtggtttttctttatttatgagatgatgaatcaatcggtctatcaagtcaacgaacagctattccgttactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 15 - DNA, 14417 bp, synthetic construct
  source 1..14417: mol_type = other DNA
ggggaattgtgagcggataacaattcccctctagaaataattttgtttaactttaagaaggagatataccatggcaatgaaaacagtagttattattgatgcattacgaacaccaattggaaaatataaaggcagcttaagtcaagtaagtgccgtagacttaggaacacatgttacaacacaacttttaaaaagacattccactatttctgaagaaattgatcaagtaatctttggaaatgttttacaagctggaaatggccaaaatcccgcacgacaaatagcaataaacagcggtttgtctcatgaaattcccgcaatgacggttaatgaggtctgcggatcaggaatgaaggccgttattttggcgaaacaattgattcaattaggagaagcggaagttttaattgctggcgggattgagaatatgtcccaagcacctaaattacaacgatttaattacgaaacagaaagctacgatgcgcctttttctagtatgatgtacgatgggttaacggatgcctttagtggtcaagcaatgggcttaactgctgaaaatgtggccgaaaagtatcatgtaactagagaagagcaagatcaattttctgtacattcacaattaaaagcagctcaagcacaagcagaagggatattcgctgacgaaatagccccattagaagtgtcaggaacgcttgtggagaaagatgaagggattcgccctaattcgagcgttgagaagctaggaacgcttaaaacagtttttaaagaagacggtactgtaacagcagggaatgcatcaaccattaatgatggggcttctgctttgattattgcttcacaagaatatgccgaagcacacggtcttccttatttagctattattcgagacagtgtggaagtcggtattgatccagcctatatgggaatttcgccgattaaagccattcaaaaactgttagcgcgcaatcaacttactacggaagaaattgatctgtatgaaatcaacgaagcatttgcagcaacttcaatcgtggtccaaagagaactggctttaccagaggaaaaggtcaacatttatggtggcggtatttcattaggtcatgcgattggtgccacaggtgctcgtttattaacgagtttaagttatcaattaaatcaaaaagaaaagaaatatggagtggcttctttatgtattggcggtggcttaggactcgctatgctactagagagacctcagcaaaaaaaaaacagccgattttatcaaatgagtcctgaggaacgcctggcttctcttcttaatgaaggccagatttctgctgatacaaaaaaagaatttgaaaatacggctttatcttcgcagattgccaatcatatgattgaaaatcaaatcagtgaaacagaagtgccgatgggcgttggcttacatttaacagtggacgaaactgattatttggtaccaatggcgacagaagagccctcagtgattgcggctttgagtaatggtgcaaaaatagcacaaggatttaaaacagtgaatcaacaacgcttaatgcgtggacaaatcgttttttacgatgttgcagatcccgagtcattgattgataaactacaagtaagagaagcggaaatttttcaacaagcagagttaagttatccatctatcgtaaaacggggcggcggcttaagagatttgcagtatcgtgcttttgatgaatcatttatatctgtcgactttttagtagatgttaaggatgcaatgggggcaaatatcgttaacgctatgttggaaggtgtggtcgagttgttccgtgaatggtttgcggagcaaaaaattttattcagtattttaagtaattatgccacggagtcggttgttacgatgaaaacggctattccagtttcacgtttaagtaaggggagcaatggccgggaaattgctgaaaaaattgttttagcttcacgctatgcttcattagatccttatcgggcagtcacgcataacaaaggaatcatgaatggcattgaagctgtagttttagctacaggaaatgatacacgcgctgttagcgcttcttgtcatgcttttgcggtgaaggaaggtcgctaccaaggcttgactagttggacgctggatggcgaacaactaattggtgaaatttcagttccgcttgctttagccacggttggcggtgccacaaaagtcttacctaaatctcaagcagccgctgatttgttagcagtgacggatgcaaaagaactaagtcgagtagtagcggctgttggtttggcacaaaatttagcggcgttacgggccttagtctctgaaggaattcaaaaaggacacatggctctacaagcacgttctttagcgatgacggtcggagctactggtaaagaagttgaggcagtcgctcaacaattaaaacgtcaaaaaacgatgaaccaagaccgagccttggctattttaaatgatttaagaaaacaataatcacgcttccgttaaggagattttatgctcaaattcagcaaaattgaaaaactgttgaggaataatatggtttcatgttctgcgcccgggaaaatctatcttttcggagaacatgcggttgtttacggagaaaccgcaatagcgtgtgcagtagagttaaggacccgggtgcgggcggagttaaatgactccataactatccagtctcagatcggcaggacaggtcttgattttgaaaaacatccctatgtctctgcagtgattgaaaaaatgagaaaatctatccccataaatggtgtttttttaactgttgattccgacattcctgttgggtcagggctcggctcatctgctgctgttacgattgcaagcataggagctctcaacgaacttttcggattcgggctttcgcttcaggaaattgcgaaactggggcatgaaattgagataaaagttcagggtgcagcgagccctactgacacctatgtttctactttcggaggagtcgttaccatccctgaaaggagaaagcttaagactcctgactgtggaattgttataggggacaccggagttttttcttctacaaaagagcttgtggcaaacgtcaggcagctccgcgaaagttaccctgatcttatcgaacctcttatgacttctattggcaaaatctccagaatcggtgagcaacttgtactttccggggactatgcttctattggcaggcttatgaatgtaaatcagggactgcttgatgcacttggagttaatatccttgagctttcacagcttatctattctgcaagggcagcaggagctttcggggcaaaaattactggagcgggaggcggtggttgtatggttgcgctaactgcaccggagaaatgtaatcaggtagcggaagccattgcaggtgcggggggcaaagtgaccattacaaaacccacggaacaggggttgaaggtcgattgaaagaaatttgcaggatacaggaataatataatgcataacgacaaagatctctctacgtggcagacattccgccgactgtggccaaccattgcgcctttcaaagcgggtctgatcgtggcgggcgtagcgttaatcctcaacgcagccagcgataccttcatgttatcgctccttaagccacttcttgatgatggctttggtaaaacagatcgctccgtgctggtgtggatgccgctggtggtgatcgggctgatgattttacgtggtatcaccagctatgtctccagctactgtatctcctgggtatcaggaaaggtggtaatgaccatgcgtcgccgcctgtttggtcacatgatgggaatgccagtttcattctttgacaaacagtcaacgggtacgctgttgtcacgtattacctacgattccgaacaggttgcttcttcttcttccggcgcactgattactgttgtgcgtgaaggtgcgtcgatcatcggcctgttcatcatgatgttctattacagttggcaactgtcgatcattttgattgtgctggcaccgattgtttcgattgcgattcgcgttgtatcgaagcgttttcgcaacatcagtaaaaacatgcagaacaccatggggcaggtgaccaccagcgcagaacaaatgctgaagggccacaaagaagtattgattttcggtggtcaggaagtggaaacgaaacgctttgataaagtcagcaaccgaatgcgtcttcaggggatgaaaatggtttcagcctcttccatctctgatccgatcattcagctgatcgcctctttggcgctggcgtttgttctgtatgcggcgagcttcccaagtgtcatggatagcctgactgccggtacgattaccgttgttttctcttcaatgattgcactgatgcgtccgctgaaatcgctgaccaacgttaacgcccagttccagcgcggtatggcggcttgtcagacgctgtttaccattctggacagtgagcaggagaaagatgaaggtaagcgcgtgatcgagcgtgcgactggcgacgtggaattccgcaatgtcacctttacttatccgggacgtgacgtacctgcattgcgtaacatcaacctgaaaattccggcagggaagacggttgctctggttggacgctctggttcgggtaaatcaaccatcgccagcctgatcacgcgtttttacgatattgatgaaggcgaaatcctgatggatggtcacgatctgcgcgagtataccctggcgtcgttacgtaaccaggttgctctggtgtcgcagaatgtccatctgtttaacgatacggttgctaacaacattgcttacgcacggactgaacagtacagccgtgagcaaattgaagaagcggcgcgtatggcctacgccatggacttcatcaataagatggataacggtctcgatacagtgattggtgaaaacggcgtgctgctctctggcggtcagcgtcagcgtattgctatcgctcgagccttgttgcgtgatagcccgattctgattctggacgaagctacctcggctctggataccgaatccgaacgtgcgattcaggcggcactggatgagttgcagaaaaaccgtacctctctggtgattgcccaccgcttgtctaccattgaaaaggcagacgaaatcgtggtcgtcgaggatggtgtcattgtggaacgcggtacgcataacgatttgcttgagcaccgcggcgtttacgcgcaacttcacaaaatgcagtttggccaatgaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgacaaagtatgcattagtcggtgatgtgggcggcaccaacgcacgtcttgctctgtgtgatattgccagtggtgaaatctcgcaggctaagacctattcagggcttgattaccccagcctcgaagcggtcattcgcgtttatcttgaagaacataaggtcgaggtgaaagacggctgtattgccatcgcttgcccaattaccggtgactgggtggcgatgaccaaccatacctgggcgttctcaattgccgaaatgaaaaagaatctcggttttagccatctggaaattattaacgattttaccgctgtatcgatggcgatcccgatgctgaaaaaagagcatctgattcagtttggtggcgcagaaccggtcgaaggtaagcctattgcggtttacggtgccggaacggggcttggggttgcgcatctggtccatgtcgataagcgttgggtaagcttgccaggcgaaggcggtcacgttgattttgcgccgaatagtgaagaagaggccattatcctcgaaatattgcgtgcggaaattggtcatgtttcggcggagcgcgtgctttctggccctgggctggtgaatttgtatcgcgcaattgtgaaagctgacaaccgcctgccagaaaatctcaagccaaaagatattaccgaacgcgcgctggctgacagctgcaccgattgccgccgcgcattgtcgctgttttgcgtcattatgggccgttttggcggcaatctggcgctcaatctcgggacatttggcggcgtgtttattgcgggcggtatcgtgccgcgcttccttgagttcttcaaagcctccggtttccgtgccgcatttgaagataaagggcgctttaaagaatatgtccatgatattccggtgtatctcatcgtccatgacaatccgggccttctcggttccggtgcacatttacgccagaccttaggtcacattctgtaagaaaaagcacaaaactggggagggatatatgcctgacgctaaaaaacaggggcggtcaaacaaggcaatgacgtttttcgtctgcttccttgccgctctggcgggattactctttggcctggatatcggtgtaattgctggcgcactgccgtttattgcagatgaattccagattacttcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaaacgttgaagtactgattctttcccgcgttctgctggggctggcggtgggtgtggcctcttataccgcaccactgtacctctctgaaattgcgccggaaaaaattcgcggcagtatgatctctatgtatcagttgatgatcactatcggaatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaatagcaacttccgccgcgcggtgttccttggcgtactgttacaggtaatgcagcaattcaccgggatgaacgtcatcatgtattacgcgccgaaaatcttcgaactggcgggttataccaacaccaccgagcaaatgtgggggacagtgattgtcggcctgaccaacgtacttgccacctttatcgcaatcggccttgttgaccgctggggacgtaaaccaacgctaacgctgggcttcctggtgatggctgctggtatgggcgtactcggtacaatgatgcatatcggtatccactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccaccgccaccaactggattgccaacatgatcgttggcgcaacgttcctgaccatgctcaacacgctgggtaacgccaacaccttctgggtgtacgcggctctgaacgtactgtttatcctgctgacattgtggctggtaccggaaaccaaacacgtttcgctggaacatattgaacgtaatctgatgaaaggtcgtaaactgcgcgaaatcggcgctcacgattaattgtacactgagaacgagacctcacacatctaacggagcgtattatatgaagaaattgctccccattcttatcggcctgagcctttctgggttcagttcgttgagccaggccgagaacctgatgcaagtttatcagcaagcacgccttagtaacccggaattgcgtaagtctgccgccgatcgtgatgctgcctttgaaaaaattaatgaagcgcgcagtccattactgccacagctaggtttaggtgcagattacacctatagcaacggctaccgcgacgcgaacggcatcaactctaacgcgaccagtgcgtccctgcagttaactcaatccatttttgatatgtcgaaatggcgtgcgttaacgctgcaggaaaaagcagcagggattcaggacgtcacgtatcagaccgatcagcaaaccttgatcctcaacaccgcgaccgcttatttcaacgtgttgaatgctattgacgttctttcctatacacaggcacaaaaagaagcgatctaccgtcaattagatcaaaccacccaacgttttaacgtgggcctggtagcgatcaccgacgtgcagaacgcccgcgcacagtacgataccgtgctggcgaacgaagtgaccgcacgtaataaccttgataacgcggtagagcagctgcgccagatcaccggtaactactatccggaactggctgcgctgaatgtcgaaaactttaaaaccgacaaaccacagccggttaacgcgctgctgaaagaagccgaaaaacgcaacctgtcgctgttacaggcacgcttgagccaggacctggcgcgcgagcaaattcgccaggcgcaggatggtcacttaccgactctggatttaacggcttctaccgggatttctgacacctcttatagcggttcgaaaacccgtggtgccgctggtacccagtatgacgatagcaatatgggccagaacaaagttggcctgagcttctcgctgccgatttatcagggcggaatggttaactcgcaggtgaaacaggcacagtacaactttgtcggtgccagcgagcaactggaaagtgcccatcgtagcgtcgtgcagaccgtgcgttcctccttcaacaacattaatgcatctatcagtagcattaacgcctacaaacaagccgtagtttccgctcaaagctcattagacgcgatggaagcgggctactcggtcggtacgcgtaccattgttgatgtgttggatgcgaccaccacgttgtacaacgccaagcaagagctggcgaatgcgcgttataactacctgattaatcagctgaatattaagtcagctctgggtacgttgaacgagcaggatctgctggcactgaacaatgcgctgagcaaaccggtttccactaatccggaaaacgttgcaccgcaaacgccggaacagaatgctattgctgatggttatgcgcctgatagcccggcaccagtcgttcagcaaacatccgcacgcactaccaccagtaacggtcataaccctttccgtaactgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

(19) United States
(12) Patent Application Publication
(10) Pub. No.: US 2024/0287548 A1
(43) Pub. Date: Aug. 29, 2024

(54) METHOD FOR CONSTRUCTING A RECOMBINANT BACTERIUM WITH HIGH PRODUCTIVITY OF BETA-ELEMENE AND GERMACRENE A

(71) Applicant: Jiangnan University, Wuxi City (CN)

(72) Inventors: Chun-Li LIU, Wuxi City (CN); Eric FORDJOUR, Wuxi City (CN); Zhonghu BAI, Wuxi City (CN); Yunpeng HAO, Wuxi City (CN); Yankun YANG, Wuxi City (CN); Xiuxia LIU, Wuxi City (CN); Ye LI, Wuxi City (CN); Jinling ZHAN, Wuxi City (CN)

(21) Appl. No.: 18/322,618

(22) Filed: May 24, 2023

(30) Foreign Application Priority Data
Feb. 28, 2023 (CN) 202310178204.3

(51) Int. Cl. C12P 5/00; C12N 9/02; C12N 9/04; C12N 9/10; C12N 9/12; C12N 15/52

(52) U.S. Cl. CPC C12P 5/002; C12N 9/0006; C12N 9/0008; C12N 9/0093; C12N 9/1022; C12N 9/1085; C12N 9/1205; C12N 9/1294; C12N 15/52; C12Y 101/01028; C12Y 101/01049; C12Y 102/03003; C12Y 102/07001; C12Y 117/07001; C12Y 202/01007; C12Y 205/0101; C12Y 207/01002; C12Y 207/09002

(57) ABSTRACT

The present invention relates to a method for constructing a recombinant bacterium with high production of β-elemene and germacrene A. Firstly, β-elemene and germacrene A are synthesized from scratch through the screening of germacrene A synthase and the overexpression of the mevalonate pathway; then, the availability of acetyl-CoA, pyruvate, and glyceraldehyde-3-phosphate in the farnesyl diphosphate pathway is ensured by deleting competing pathways in the central carbon metabolism; next, the present invention uses lycopene color as a high-throughput screening method and obtains an optimized NSY305N through error-prone PCR. Finally, in shake flasks, strain β-EL-4 constructed through key pathway enzymes, efflux engineering, and translation engineering produced 1161.09 mg/L of β-elemene and 852.36 mg/L of germacrene A, which is the highest reported yield at shake flask level. In 4-L fed-batch fermentation, the production of β-elemene and germacrene A reached 3.52 g/L and 2.13 g/L, respectively.

### CROSS-REFERENCE TO RELATED APPLICATIONS

The present application claims the priorities from the Chinese patent application number 2023101782043 filed Feb. 28, 2023, and the disclosure of which is incorporated herein by reference in its entirety.

### REFERENCE TO SEQUENCE DISCLOSURE

The sequence listing file under the file name “P2732US00_Sequence_listing.xml” submitted in ST.26 XML file format with a file size of 67 KB created on May 5, 2023 is incorporated herein by reference.

### FIELD OF THE INVENTION

The present invention generally relates to the technical field of genetic engineering technology. In particular, it relates to a method for constructing a recombinant bacterium with high productivity of β-elemene and germacrene A.

### BACKGROUND OF THE INVENTION

β-elemene is a naturally occurring sesquiterpene mainly isolated from plants of the Zingiberaceae family. It is one of the most widely used anti-cancer drugs for the treatment of various types of tumors and has broad application prospects and market demand. However, the current yield of β-elemene extracted from the Zingiberaceae plant Curcuma wenyujin is far from meeting industrial needs.

The chemical synthesis method for producing β-elemene is costly, has low yield, and is non-renewable. Therefore, the use of microbial chassis for green manufacturing of β-elemene production is highly anticipated. Its production efficiency involves multiple complex physiological processes, including balancing expression of metabolic pathways, protein expression efficiency, activity of key enzymes, accumulation of byproducts, and extracellular transport. Analyzing the effects and mechanisms of these physiological processes on the biosynthesis of β-elemene is crucial for constructing high-yielding microbial chassis for sesquiterpenes.

According to research, β-elemene is formed by Cope-Claisen rearrangement of germacrene A. Although β-elemene has been extracted from plants of the Zingiberaceae family, its synthetic enzyme has not been identified. Therefore, in biological research, the germacrene A synthase is used to synthesize β-elemene. Recently, a Class I non-plant sesquiterpene synthase that catalyzes FPP ionization to germacrene A was identified in Nostoc sp. PCC 7120. It has been introduced into the brewing yeast (S. cerevisiae) for germacrene A biosynthesis and achieved yields of 190.7 mg/L and 309.8 mg/L in shake flasks; Chen et al. and Li et al. achieved the production of 126.4 mg/L and 364.26 mg/L of β-olivene, respectively, by expressing the germacrene A synthase in Escherichia coli.

The efficiency of biosynthesis of isoprene involves multiple complex physiological processes, such as balancing the expression of metabolic pathways, accumulation of pathway precursors (metabolic engineering), protein expression efficiency (translation engineering), activity of key enzymes (protein engineering), and extracellular transport (efflux engineering).

Therefore, understanding the role and mechanism of β-olive synthesis is crucial for constructing a chassis. Microbial biosynthesis of β-elemene depends on the availability of the intracellular precursor farnesyl pyrophosphate (FPP), which is derived from the condensation of the two universal isoprenoid precursors, isopentenyl pyrophosphate (IPP) and dimethylallyl pyrophosphate (DMAPP). The two main pathways for the supply of IPP and DMAPP are the mevalonate (MVA) pathway and the 2-C-methyl-D-erythritol 4-phosphate (MEP) pathway. Among them, the exogenous MVA pathway has been introduced into Escherichia coli to effectively produce isoprenoids. In addition to metabolic engineering of the MVA pathway, rewriting natural central carbon metabolism is also an important strategy for effectively utilizing derived molecules at key nodes.

The present invention provides examples of successfully coordinating the use of metabolic engineering, transcriptional engineering, efflux engineering, and protein engineering in the production of highest levels of sesquiterpenes in the production of sesquiterpenes and the construction of efficient cell factories.

### SUMMARY OF THE INVENTION

This section aims to summarize some aspects of the embodiments of the present invention and to briefly describe some preferred embodiments. Simplification or omission may be made in this section, the abstract of the specification, and the title to avoid obscuring the purposes of this section, the abstract of the specification, and the title. Such simplification or omission may not be used to limit the scope of the present invention.

The present invention is made in view of the technical problems as above-mentioned.

Therefore, the object of the present invention is to overcome the shortcomings in the prior art and provide a method for constructing a recombinant bacterium with high yield of β-elemene and germacrene A.

To solve the technical problems as above-mentioned, the present invention provides the following solutions, including:
selecting a synthetic enzyme with high efficiency in de novo biosynthesis of β-elemene;
increasing the production of β-elemene by overexpressing an enzyme ispA; further increasing the production of β-elemene through RBS engineering or protein fusion technology;
enhancing a precursor accumulation by rewriting a central carbon metabolism pathway;
high-throughput screening of a directed evolution enzyme NS to obtain a highly active NS enzyme;
increasing the production of β-elemene by adjusting metabolism to recycle accumulated pyruvic acid;
adjusting an efflux pump to promote the production of β-elemene; and constructing the recombinant bacterium with high yield of β-elemene and germacrene A.

As a preferred embodiment of the method for constructing high-yield recombinant bacterium of β-elemene and germacrene A according to the present invention, where the synthetic enzyme includes β-elemene synthase genes of TS (NCBI Accession number: AB730585) from Toona sinensis, OS (NCBI Accession number: ABJ16553) from rice, and NS (NCBI Accession number: BAB76384) from Nostoc sp. PCC7120, with sequences identified as SEQ ID NO: 1, SEQ ID NO: 2, and SEQ ID NO: 3, respectively.

As a preferred embodiment of the method for constructing high-yield recombinant bacterium of β-elemene and germacrene A according to the present invention, where the step of increasing the production of β-elemene by overexpressing an enzyme ispA includes co-transforming a plasmid pRSF-INI carrying an overexpressed gene ispA of Escherichia coli with a plasmid pA-ESKKD carrying a metabolic pathway for synthesizing β-elemene and germacrene A into Escherichia coli BL21 (DE3), the sequence of the plasmid pA-ESKKD is shown as SEQ ID NO: 4.

As a preferred embodiment of the method for constructing high-yield recombinant bacterium of β-elemene and germacrene A according to the present invention, where the step of increasing the production of β-elemene through RBS engineering includes generating an electronic RBS library for the enzyme NS using an RBS calculator, and adding RBS sequences with translation initiation rates (TIR) ranging from 500 to 50,000 arbitrary units to the 5′ end of the NS to create a plasmid pRSF-INI.

As a preferred embodiment of the method for constructing high-yield recombinant bacterium of β-elemene and germacrene A according to the present invention, where the step of increasing the production of β-elemene through protein fusion technology includes fusing the enzyme ispA and the enzyme NS using four different linkers to fuse the N-terminus of type I non-plant synthase NS with the C-terminus of an endogenous ispA, the four different linkers include a short chain linker, a medium chain linker, a long chain linker, and a flexible chain linker. The short chain is GGGS, the medium chain is (GGGS)2, the long chain is (GGGS)3, and the flexible chain is GGGGS.

As a preferred embodiment of the method for constructing high-yield recombinant bacterium of β-elemene and germacrene A according to the present invention, where the step of enhancing a precursor accumulation by rewriting a central carbon metabolism pathway includes knocking out one or more genes encoding pyruvate dehydrogenase (poxB), phosphoenolpyruvate synthase (ppsA), glucose-specific phosphotransferase system enzyme (ptsG), glucose-specific IIA component of the phosphotransferase system (crr), acetaldehyde dehydrogenase (adhE), D-lactate dehydrogenase (ldhA), and glucose-6-phosphate dehydrogenase (zwf) in the wild-type strain Escherichia coli BL21 (DE3), resulting in the defective strain ΔpoxBΔppsAΔptsGΔcrrΔadhEΔldhAΔzwf; and overexpressing one or more genes encoding galactose permease (galP) and glucose kinase (glk). The sequence of knocked-out gene poxB is shown as SEQ ID NO: 5; the sequence of knocked-out gene ppsA is shown as SEQ ID NO: 6; the sequence of knocked-out gene ptsG is shown as SEQ ID NO: 7; the sequence of knocked-out gene crr is shown as SEQ ID NO: 8; the sequence of knocked-out gene adhE is shown as SEQ ID NO: 9; the sequence of knocked-out gene ldhA is shown as SEQ ID NO: 10; the sequence of knocked-out gene zwf is shown as SEQ ID NO: 11. The plasmids used for gene knockout are pCas9 and pTarget.

As a preferred embodiment of the method for constructing high-yield recombinant bacterium of β-elemene and germacrene A according to the present invention, where the step of increasing the production of β-elemene by adjusting metabolism to recycle accumulated pyruvic acid includes cloning 1-deoxy-D-xylulose-5-phosphate synthase (dxs), 1-hydroxy-2-methyl-2-(E)-butenyl 4-diphosphate synthase (ispG), and pforA into the plasmid pRSF-INI to generate plasmids of pRSF-INIDG and pRSF-IN*IA, respectively, the plasmids are then expressed in the defective strain ΔpoxBΔppsAΔptsGΔcrrΔadhEΔldhAΔzwf along with plasmids of pA-ESKKD and pET-EKGG; where the sequence of gene dxs is shown as SEQ ID NO: 12; the sequence of gene ispG is shown as SEQ ID NO: 13; the sequence of plasmid pRSF-IN*IA is shown as SEQ ID NO: 14.

As a preferred embodiment of the method for constructing high-yield recombinant bacterium of β-elemene and germacrene A according to the present invention, where the step of adjusting an efflux pump to promote the production of β-elemene comprises transferring plasmids of pET-EKMGGT and pA-ESKKD overexpressing an outer membrane protein tolC and an inner membrane lipopolysaccharide msbA of the efflux pump into the defective strain ΔpoxBΔppsAΔptsGΔcrrΔadhEΔldhAΔzwf along with plasmids of pRSF-IN*IDG and pRSF-IN*IA, respectively. The sequence of plasmid pET-EKMGGT is shown as SEQ ID NO: 15.

As a preferred embodiment of the method for constructing high-yield recombinant bacterium of β-elemene and germacrene A according to the present invention, where the recombinant bacterium obtained by the method produces 1161.09 mg/L of β-elemene and 852.36 mg/L of germacrene A through fermentation in a shaking flask.

As a preferred embodiment of the method for constructing high-yield recombinant bacterium of β-elemene and germacrene A according to the present invention, where the recombinant bacterium obtained by the method produces 3.52 g/L of β-elemene and 2.13 g/L of germacrene A in a 4-L fed-batch fermentation.

The present invention has the following advantages:
(1) The present invention describes a Escherichia coli cell factory that produces high levels of β-elemene and germacrene A. By deleting competing pathways in the central carbon metabolism, the availability of acetyl-CoA, pyruvate, and glyceraldehyde-3-phosphate in the farnesyl diphosphate pathway is ensured. The present invention uses lycopene color as a high-throughput screening method and obtains an optimized NS mutant Y305N through error-prone PCR. In shake flasks, strain β-EL-4 constructed through key pathway enzymes, efflux engineering, and translation engineering produced 1161.09 mg/L of β-elemene and 852.36 mg/L of germacrene A, which is the highest reported yield at shake flask level. In 4-L fed-batch fermentation, the production of β-elemene and germacrene A reached 3.52 g/L and 2.13 g/L, respectively.
(2) The present invention has elucidated the biosynthetic and regulatory mechanisms of sesquiterpenes during the process of constructing a high-yield β-elemene-producing chassis cell. This process is applicable for microbial production of a wider range of chemicals and also indicates the feasibility of central metabolism engineering of recombinant Escherichia coli for cost-effective, large-scale production of acetyl-CoA and pyruvate-derived compounds. The techniques and strategies developed in this study provide a successful example and reference for microbial production of a wider range of terpenoid compounds.

### BRIEF DESCRIPTION OF THE DRAWINGS

In order to more clearly illustrate the technical solutions of the embodiments of the present invention, the following will briefly introduce the drawings needed to be used in the description of the embodiments. Obviously, the drawings in the following description are only some embodiments of the present invention. For those of ordinary skill in the art, other drawings can be obtained based on these drawings without any creative labor, in which:

FIG. 1 depicts a biosynthetic pathway of β-elemene;

FIG. 2 depicts a construction map of plasmid pA-ESKKD in the present invention;

FIG. 3 depicts a construction map of plasmid pRSF-IN*IA in the present invention;

FIG. 4 depicts a construction map of the plasmid pET-EKMGG in the present invention.

FIGS. 5A-5D depict a schematic diagram of the process of de novo synthesis of β-elemene in Escherichia coli in the present invention;

FIG. 6 depicts a relevant schematic diagram of using multiple synthetic RBS sequences to regulate the translation of NS, and fusion protein technology to improve β-elemene production in the present invention;

FIG. 7 depicts a relevant schematic diagram of rewriting central carbon metabolic pathways to reduce byproduct accumulation and increase precursor accumulation in the present invention;

FIG. 8 depicts a schematic diagram of high-throughput screening and directed evolution of the NS enzyme to obtain highly active NS enzyme in the present invention;

FIG. 9 depicts shows the verification results of the determination of lycopene production in Example 8 of the present invention;

FIG. 10 depicts docking model between residue Y305 and N305 and the ligand in Example 8 of the present invention;

FIG. 11 depicts a schematic diagram of adjusting metabolism to recycle accumulated pyruvic acid to increase the production of β-elemene; and

FIG. 12 depicts a schematic diagram of the fed-batch fermentation process for β-elemene in Example 11 of the present invention.

### DETAILED DESCRIPTION

To make the above-mentioned objectives, features and advantages more easily be understood, the following detailed description of the embodiments of the present invention is provided in conjunction with the specification.

Although the following descriptions illustrate in detail in order to facilitate understanding of the present invention, it should be understood by a skilled person in the art that the present invention can also be enabled by other ways not described herein. The skilled person in the art can also implement the present invention without departing from the spirit of the present invention such that the following descriptions concerning the examples will not limit the present invention.

In addition, the expressions “an embodiment” or “an example” used herein refers to including specific features, structure and characteristics of at least one embodiment of the present invention. “According to an embodiment of the present invention” appears in the present disclosure does not necessarily mean that it refers to the same embodiment, or it does not necessarily mean that it independently or selectively contradicts with one another.

Measurement of β-elemene, germacrene A and farnesol (FOH) concentrations in the present invention:
(1) identification of β-linalool and farnesol was performed using a GC-MS system (Shimadzu Co., Kyoto, Japan) equipped with an RTX-5MS column (30 m×0.32 mm×0.25 μm);
(2) each 1 microliter sample of dodecane was injected into the system at a split ratio of 1:10, and set the carrier gas helium to a constant flow rate of 0.78 mL/min;
(3) the column temperature was initially maintained at 40° C. for 2 minutes, and then gradually increased at a rate of 10° C./min to 160° C. and held for 2 minutes, and finally increased at a rate of 15° C./min to 300° C. and held for 5 minutes. The mass spectrometer was set in SCAN mode to scan m/z ions in the range of 40-250 for the identification of β-elemene;
(4) β-elemene standard solutions were prepared in different concentrations of dodecane solvent to calibrate the standard curve. At the same time, the standard curve of β-elemene was used for quantifying germacrene A.

Determination of organic acid (alcohol) and glucose concentrations in the present invention: the concentrations of acetic acid, lactic acid, succinic acid, ethanol, and glucose were analyzed using an Agilent 1200 series high-performance liquid chromatography (HPLC) system equipped with a refractive index detector and a UV detector. The sample was separated on an Aminex HP-87H column (300×7.8 mm, Biorad) at 35° C. Under isothermal conditions, organic compounds were separated using a 5 mM H₂SO₄ solution at a constant flow rate of 0.5 mL/min. Standard solutions of pyruvic acid, lactic acid, acetic acid, succinic acid, ethanol, and glucose were prepared in water at different concentrations to make standard curves.

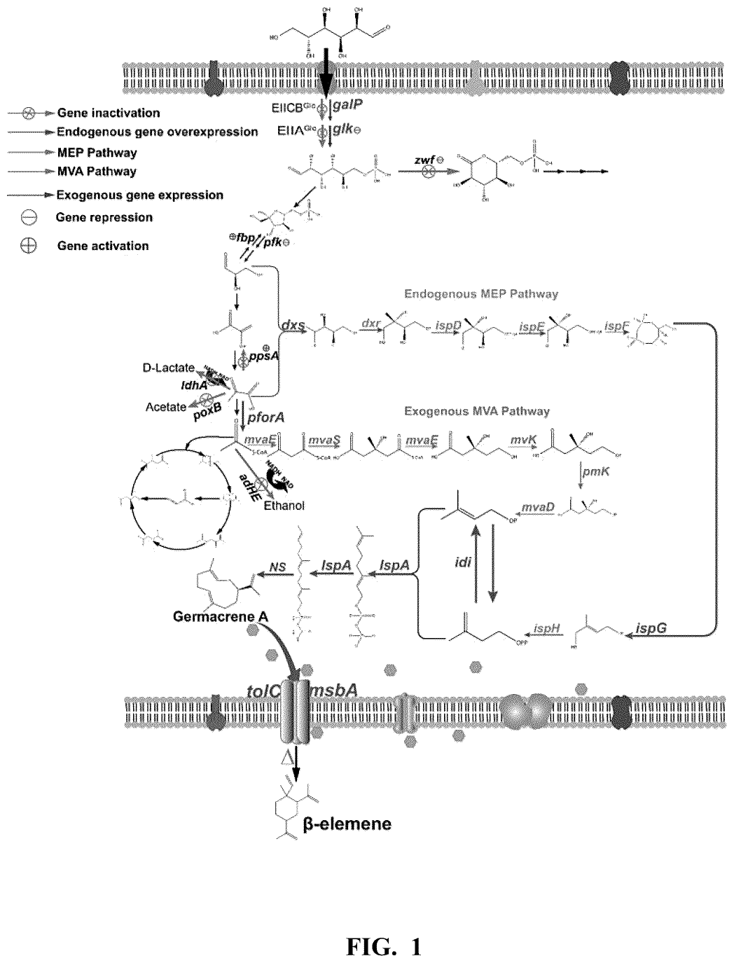

Measurement of intracellular lycopene concentration in the present invention:
(1) the 2 mL sample was centrifuged at 10,000×g for 5 minutes to pellet the cells;
(2) the cells were washed twice with double-distilled water, centrifuged again, and resuspended in 2 mL of acetone. The suspension was incubated in the dark at 55° C. with intermittent vortexing for 15 minutes, and then centrifuged at 8000×g for 3 minutes;
(3) the acetone extracts were measured at OD472 nm on a spectrophotometer, using acetone as blank, to indirectly determine the relative lycopene content; (4) the continuous dilution of standard stock solution was used to create a standard curve.
FIG. 1 is the biosynthesis route of β-elemene in the present invention, including, endogenous MEP approach and exogenous MVA approach. Eliminating the competitive pathway will increase the accumulation of downstream β-elemene biosynthesis precursors. The sequence of germacrene A synthase was codon-optimized for expression in Escherichia coli BL21 (DE3).

The specific embodiments are as follows:

### EXAMPLES

### Example 1

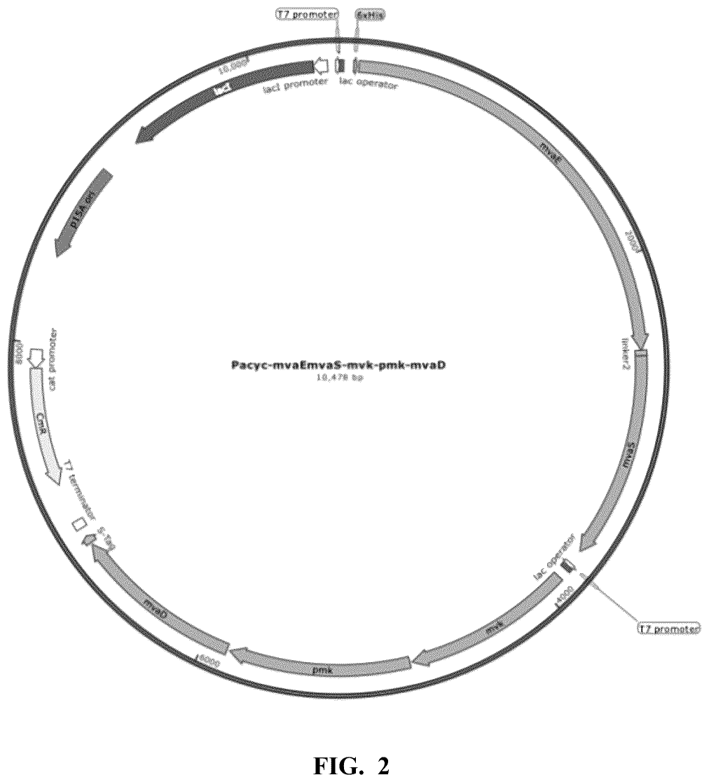

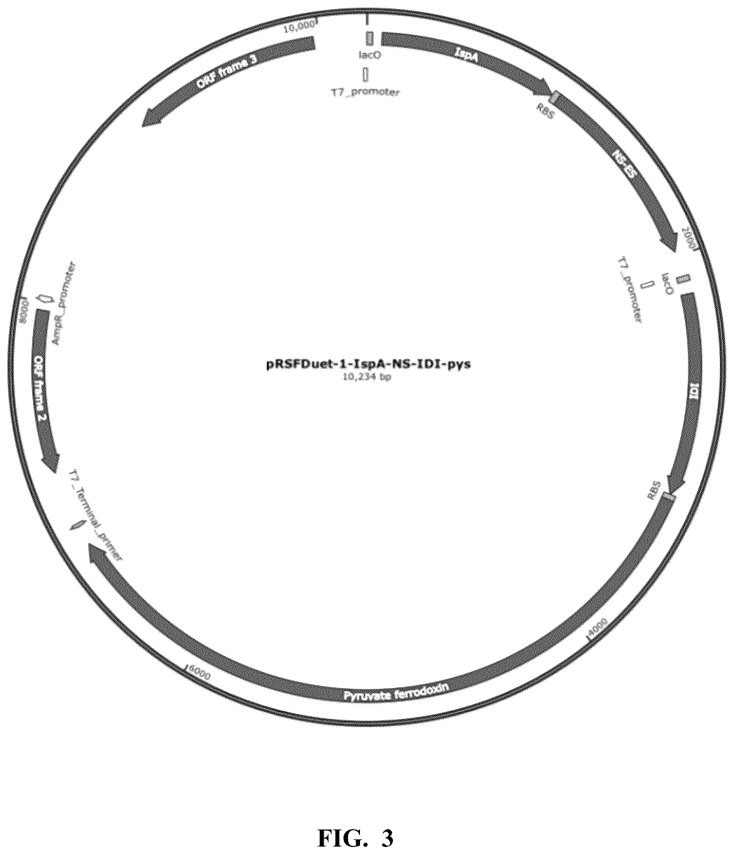

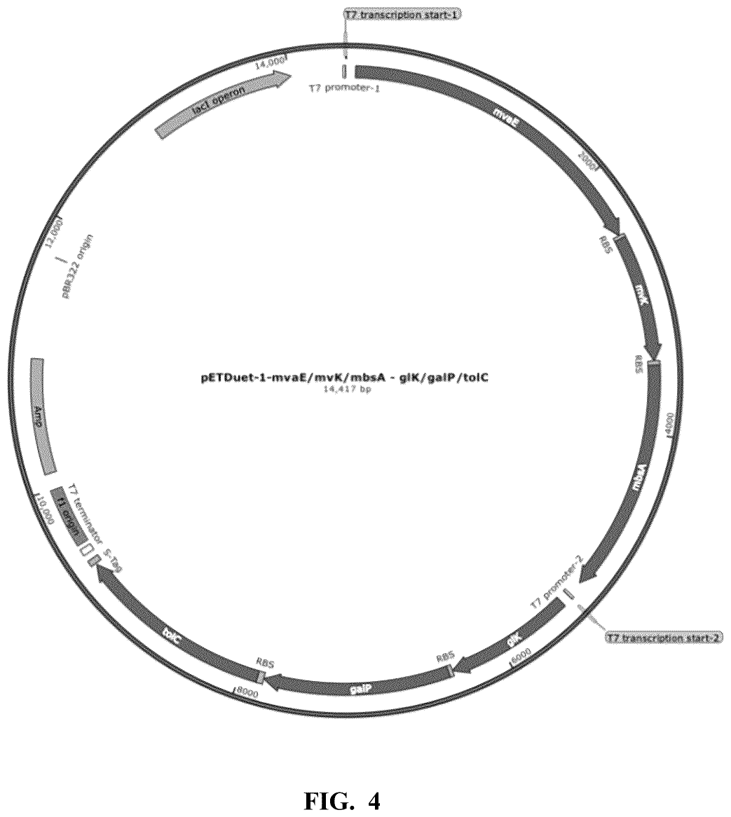

Referring to FIGS. 2-4, the construction diagrams of plasmids pA-ESKKD, pRSF-IN*IA, and pET-EKMGGT are shown, respectively. This example provides a method for constructing plasmids carrying metabolic pathways for the synthesis of β-elemene and germacrene A in the process of constructing a recombinant bacterium with high productivity of β-elemene and germacrene A. The specific steps are as follows:
(1) Suzhou Jinweizhi Biotechnology Co., Ltd. was commissioned to synthesize germacrene A synthase from Toona sinensis (Genebank: AB730585), Oryza sativa Japonica (Genebank: ABJ16553), and Nostoc sp. PCC 7120 (Genebank: BAB76384) with codon optimization. Clone the synthase with primers NS-F and NS-R into the NcoI/BamHI digested pRSFDuet-1 plasmid, and obtain plasmids pRS-NS, pRS-TS, and pRS-OS through digestion, ligation, and sequencing validation;
(2) The idi gene from Streptococcus pneumoniae was cloned into pRS-NS, pRS-TS, and pRS-OS at the NdeI/XhoI sites using primers IDI-NS-F and IDI-R. After digestion, ligation, and sequencing verification, the plasmids pRS-NS-idi, pRS-TS-idi, and pRS-OS-idi were obtained;
(3) The endogenous ispA gene was cloned into the NcoI/BamHI sites of pRS-NS-IDI using primers ispA-F and ispA-R. After enzymatic digestion, Gibson assembly and sequencing verification, the plasmid pRS-NS-ispA-IDI was obtained;
(4) The NS(Y305N) mutant gene was replaced with the NS gene in the plasmid pRS-NS-ispA-IDI using Gibson assembly with primers NS*-F and NS*-R to obtain the plasmid pRS-NS*-ispA-IDI;
(5) Pyruvate ferredoxin oxidoreductase (pforA) was cloned using primers pforA-F and pforA-R into the NdeI/XhoI sites of plasmid pRS-NS-ispA-IDI, and verify the construct by enzymatic digestion, ligation, and sequencing. The resulting plasmid is named pRS-NS-ispA-IDI-pforA;
(6) The 1-deoxy-D-xylulose 5-phosphate synthase (dxs) and 1-hydroxy-2-methyl-2-(E)-butenyl 4-diphosphate synthase (ispG) genes were cloned into the NdeI/XhoI sites of the plasmid pRS-NS-ispA-IDI using the primers dxs-F, dxs-R, ispG-F, and ispG-R. The resulting plasmid was verified by enzymatic digestion, ligation, and sequencing, and named pRS-NS-ispA-IDI-dxs-ispG;
(7) The genes encoding glucose kinase (glk) and galactose permease (galP) were inserted into the NdeI/XhoI sites of the pETDuet-1 plasmid using the Gibson assembly method, with the primers glk-F and glk-R, and galP-F and galP-R, resulting in the plasmid pET-glk-galP (pET-GG);
(8) The genes 3-hydroxy-3-methylglutaryl coenzyme A synthase (mvaE) and mevalonate kinase (mvk) were inserted into the NcoI/BamHI sites of the plasmid pET-glk-galP (pET-GG) using Gibson assembly with the primers mvaE-F and mvaE-R, and mvk-F and mvk-R. The resulting plasmid was named pET-mvaE-mvk-glk-galP (pET-MMGG). The primer sequences mvaE-F and mvaE-R, mvk-F and mvk-R are shown in Table 1;
(9) The genes for lipid ABC transporter permease (msbA) and outer membrane protein transporter channel (tolC) were inserted into the NcoI/BamHI and NdeI/XhoI sites of the plasmid pET-mvaE-mvk-glk-galP (pET-MMGG), respectively, using Gibson assembly with primers msbA-F and msbA-R, tolC-F and tolC-R. The resulting plasmid was named pET-mvaE-mvk-msbA-glk-galP-tolC (pET-EKMGGT).

The sequences of the plasmids used in the above plasmid construction process are shown in Table 1.

TABLE 1
plasmid sequences used for plasmid construction

| Primer | Sequence |
|---|---|
| NS-F | TTTGTTTAACTTTAATAAGGAGATATACCATGGGCATGGA |
| | AAAAATTACCTTTCCGAACC |
| NS-R | GTCGTCATGTTACCCCTTGGTTGACTCGAATGGGTCTTTA |
| | GCTCGCCATCAGTTCCAGTT |
| IDI-NS-F | GAGCTAAAGACCCATTCGAGTCAACCAAGGGGTAACATGA |
| | CGACAAATCGTAAGGACGAG |
| OS-F | ATAATTTTGTTTAACTTTAATAAGGAGATATACCATGGGC |
| | ATGGCGACGAGCGTGCCAA |
| OS-R | GTCGTCATGTTACCCCTTGGTTGACTCGAATGGGTCTTTA |
| | CACGCTCAGAATATAAATGCTATGAA |
| IDI-OS-F | CGTGTAAAGACCCATTCGAGTCAACCAAGGGGTAACATGA |
| | CGACAAATCGTAAGGACGAG |
| TS-F | ATAATTTTGTTTAACTTTAATAAGGAGATATACCATGGGC |
| | ATGAGCGTGCCGGTGAGTCA |
| TS-R | GTCGTCATGTTACCCCTTGGTTGACTCGAATGGGTCTTTA |
| | CACCGGAATCGGATCAATCA |
| IDI-TS-F | GGTGTAAAGACCCATTCGAGTCAACCAAGGGGTAACATGA |
| | CGACAAATCGTAAGGACGAG |
| IDI-R | CGACCTGCAGGCGCGCCGAGCTCGAATTCGGATCCTTACG |
| | CCTTTTTCATCTGATCATTT |
| glk-F | ATTAGTTAAGTATAAGAAGGAGATATACATATGACAAAGT |
| | ATGCATTAGTCGGTGAT |
| glk-R | AGGCATATATCCCTCCCCAGTTTTGTGCTTTTTCTTACAG |
| | AATGTGACCTAAGGTCTGGC |
| galP-F | CATTCTGTAAGAAAAAGCACAAAACTGGGGAGGGATATAT |
| | GCCTGACGCTAAAAAACAGG |
| galP-R | CGTTAGATGTGTGAGGTCTCGTTCTCAGTGTACAATTAAT |
| | CGTGAGCGCCGATTTC |
| mvaE-F | TTAACTTTAAGAAGGAGATATACCATGGCAATGAAAACAG |
| | TAGTTATTATTGATGCATTA |
| mvaE-R | GAGCATAAAATCTCCTTAACGGAAGCGTGATTATTGTTTT |
| | CTTAAATCATTTAAAATAGC |
| mvk-F | AAAACAATAATCACGCTTCCGTTAAGGAGATTTTATGCTC |
| | AAATTCAGCAAAATTGAAAA |
| mvk-R | TATATTATTCCTGTATCCTGCAAATTTCTTTCAATCGACC |
| | TTCAACCCCTGT |
| tolC-F | GAACGAGACCTCACACATCTAACGGAGCGTATTATATGAA |
| | GAAATTGCTCCCCATTCT |
| tolC-R | AATTTCGCAGCAGCGGTTTCTTTACCAGACTCGAGTCAGT |
| | TACGGAAAGGGTTATGACC |
| mbsA-F | AAGAAATTTGCAGGATACAGGAATAATATAATGCATAACG |
| | ACAAAGATCTCTCTACGT |
| mbsA-R | CGACCTGCAGGCGCGCCGAGCTCGAATTCGGATCCTCATT |
| | GGCCAAACTGCATTTTG |
| dxs-F | AAGGCGTAATGAACACCTAAGGAGGAGAAAGAAATGAGTT |
| | TTGATATTGCCAAATACCC |
| dxs-R | TTATGCATATAAATCCTTCTTATTCTGAATGGAAGTTAGA |
| | TTATGCCAGCCAGGCCTTGA |
| ispG-F | CATAATCTAACTTCCATTCAGAATAAGAAGGATTTATATG |
| | CATAACCAGGCTCCAATTCA |
| ispG-R | AATTTCGCAGCAGCGGTTTCTTTACCAGACTCGAGTTATT |
| | TTTCAACCTGCTGAACGTCA |
| F1 | ATGGAAAAAATTACCTTTCCGAACC |
| R2 | CGACCTGCAGGCGCGCCGAGCTCGAATTCGGATCCTTAGC |
| | TCGCCATCAGTTCCAGTT |
| ispA-F | TAATTTTGTTTAACTTTAATAAGGAGATATACCATGGGCA |
| | TGGACTTTCCGCAGCAACTC |
| ispA-R | TTAGCTCGCCATCAGTTCCAGTT |
| pforA-F | GCGTAAACATTACGTAAGTAAGGGGGGCTAGGATGTCAAA |
| | AGTAATGAAGACAATGGAT |
| pforA-R | TTCGCAGCAGCGGTTTCTTTACCAGACTCGAGTTAATTCT |
| | GCTCAGCCAGTTTCTTGTAT |
| NS*-F | CCATGGGCATGGAAAAAATTACCTTTCCGAACCT |
| NS*-R | GGATCCTTAGCTCGCCATCAGTTCCAGTTT |

The PCR, plasmid extraction, DNA purification, ligation, PCR, and enzyme digestion were performed according to standard molecular biology protocols and kit instructions (as shown in systems in Tables 2-5). The plasmid construction was carried out using the enzyme digestion-ligation method and the Gibson assembly method. The plasmids constructed in this study and their relevant annotations are shown in Table 6.

TABLE 2
PCR reaction system

| Components | Volume | |
|---|---|---|
| Polymerase mix | 25 | μL |
| Template | 3 | μL |
| Primer_F | 2.5 | μL |
| Primer_R | 2.5 | μL |

| | |
|---|---|
| ddH₂O | up to 50 μL |

| The PCR reaction conditions are: | |
|---|---|
| 98° C. pre-denaturration 3 min | 39 Cycles |
| 98° C. denaturation 15 s | |
| 55° C. annealing 10 s | |
| 65° C. annealing 10 s | |
| 72° C. elongation 12 s | |
| 72° C. thermal insulation 5 min | |

TABLE 3
enzyme digestion reaction system

| Components | Volume | |
|---|---|---|
| DNA | 25 | μL |
| 10 × Buffer | 5 | μL |
| Enzyme 1 | 2 | μL |
| Enzyme 2 | 2 | μL |

| | |
|---|---|
| ddH₂O | up to 50 μL |

TABLE 4
enzyme ligation reaction system

| Components | Volume | |
|---|---|---|
| 10 × Buffer | 2 | μL |
| Gene fragment | 10 | μL |
| Vector | 2 | μL |
| T4 ligase | 1 | μL |

| | |
|---|---|
| ddH₂O | up to 20 μL |

TABLE 5
Gibson assembly reaction system

| Components | Volume | |
|---|---|---|
| Mix | 10 | μL |
| Gene fragment | 10 | μL |
| Vector | 2 | μL |

| | |
|---|---|
| ddH₂O | up to 20 μL |

TABLE 6
plasmids used in this study

| Plasmids | Description | Reference |
|---|---|---|
| pRSFDuet-1 | An expression vector, RSF ori, T7lac promoter, lacI gene, Kan^{r}, | Novagene |
| | two MCS | |
| pRSF-NI | pRSFDuet-1 expressing Nostoc sp terpene synthase, and idi | this study |
| pRSF-TI | pRSFDuet-1 expressing Toona sinensis terpene synthase, and idi | this study |
| pRSF-OI | pRSFDuet-1 expressing Oryza sativa Japonica terpene synthase, | this study |
| | and idi | |
| pRSF-INI | pRSFDuet-1 containing Nostoc sp terpene synthase, ispA and idi | this study |
| pR1-13 | pRSFDuet-1 containing Nostoc sp terpene synthase, ispA, idi and | this study |
| | RBS library for Nostoc sp terpene synthase | |
| pL1-4 | pRSFDuet-1 containing Nostoc sp terpene synthase, ispA, idi and | this study |
| | linker arrays for Nostoc sp terpene synthase | |
| pRSF-N*I | pRSFDuet-1 containing mutant Nostoc sp terpene synthase and | this study |
| | idi | |
| pRSF-IN*I | pRSFDuet-1 containing mutant Nostoc sp terpene synthase, ispA | this study |
| | and idi | |
| pRSF-IN*IDG | pRSFDuet-1 containing mutant Nostoc sp terpene synthase, ispA, | this study |
| | idi, dxs, ispG | |
| pRSF-IN*IA | pRSFDuet-1 containing mutant Nostoc sp terpene synthase, ispA, | this study |
| | idi, pforA | |
| pETDuet-1 | An expression vector, T7 promoter/lac operator, pBR322-derived | Novagene |
| | ColE1 replicon, lacI gene, Amp^{r}, two MCS | |
| pET-crtEBII | pETDuet-1 containing C. glutamicum lycopene pathway and idi | (in publishing) |
| | from S. cerevisiae | |
| pET-GG | pETDuet-1 expressing glK, and galP | this study |
| pET-EKGG | pETDuet-1 expressing mvaE, mvK, glK, and galP | this study |
| pET-MTGG | pETDuet-1 expressing msbA, tolC, glK and galP | this study |
| pET-EKMGGT | pETDuet-1 expressing mvaE, mvK, msbA, glK, galP and tolC | this study |
| pACYCDuet-1 | An expression vector, T7lac promoter, CloDF13-derived CDF replicon, | Novagene |
| | lacI gene, Cm^{R}, two MCS | |
| pA-ESKKD | pACYCDuet-1 expressing mvaE, mvaS, mvK, pmK, and pmkD | (Liu et al., 201 |
| | | 9b) |
| pCas | RepA101(Ts) ori, Kan^{r}, Pcas-cas9, ParaC-Red, lacIq | (Jiang et al., 20 |
| | | 15) |
| pTarget | sgRNA plasmid, pMB1 ori, Spe^{r} | (Jiang et al., 20 |
| | | 15) |
| pTarget-poxB | pTarget expressing sgRNA-poxB | this study |
| pTarget-ppsA | pTarget expressing sgRNA-ppsA | this study |
| pTarget-ptsG | pTarget expressing sgRNA-ptsG | this study |
| pTarget-crr | pTarget expressing sgRNA-crr | this study |
| pTarget-adhE | pTarget expressing sgRNA-adhE | this study |
| pTarget-ldhA | pTarget expressing sgRNA-ldhA | this study |
| pTarget-zwF | pTarget expressing sgRNA-zwF | this study |

### Example 2

The purpose of this embodiment is to obtain a series of gene knockout mutants based on wild-type Escherichia coli BL21 (DE3). The present invention utilizes a dual-plasmid CRISPR-Cas9 system to delete the genes encoding pyruvate oxidase (poxB), phosphoenolpyruvate synthase (ppsA), glucose-specific phosphotransferase system enzyme (ptsG), glucose-specific IIA component of phosphotransferase system (crr), acetaldehyde dehydrogenase (adhE), D-lactate dehydrogenase (ldhA), and glucose-6-phosphate dehydrogenase (zwf). The specific steps are as follows:
(1) Design sgRNA (a 20-nucleotide sequence complementary to the target gene) using software such as sgRNA Scorer (Genscript, 2015) as an example. A series of corresponding single guide RNAs (sgRNAs) were designed for the target genes poxB, ppsA, ptsG, crr, adhE, ldhA, and zwf. The sequences of poxB-sgRNA, ppsA-sgRNA, ptsG-sgRNA, crr-sgRNA, adhE-sgRNA, ldhA-sgRNA, zwf-sgRNA are shown in Table 7 below;

TABLE 7
sgRNA for gene knockout

| Target gene | N20 | PAM |
|---|---|---|
| poxB | AATTGGCAGCGGCTATTTCC | AGG |
| ppsA | ATGGGTGTTTCCGTTCCGAA | TGG |
| ptsG | GTATCCGTACTGCCTATCGC | AGG |
| crr | CAGATTCGATAGAGAATGCG | TGG |
| adhE | TCGAATCCCACTCGCGAAAA | TGG |
| ldhA | TGGTCAGAGCTTCTGCTGTC | AGG |
| zwf | GCCAGTTATCAATGTCGACG | CGG |

(2) The sgRNA of poxB, ppsA, ptsG, crr, adhE, ldhA, zwf genes were inserted into the pTarget plasmid respectively;
(3) The following procedure was carried out to blunt the kinases to ensure incompatible end ligation:
   a. Linear DNA 1.3 mg(x);
   b. OX Blunting Kination Buffer 1 uL;
   c. Blunting Kination Enzyme Mix 0.5 uL;
   d. Adding ddH₂0 to 10 uL (10-x);
   e. Incubating at 37° C. for 10 minutes, and then at 70° C. for 10 minutes in a thermal cycler;
   f. Ligation reaction mixture: 5 uL of the reaction mixture and 5 uL of solution I (Takara);
   g. Incubating at 16° C. for 12 to 16 hours;
   h. Transforming the plasmid and spreading it on a LB agar plate containing ampicillin.
(4) Designing and constructing donor genes. include amplifying the target genes. The primers used for amplifying the upstream and downstream sequences of poxB, ppsA, ptsG, crr, adhE, ldhA, and zwf genes are as follows: poxB-U-F, poxB-U-R, poxB-D-F, poxB-D-R, ppsA-U-F, ppsA-U-R, ppsA-D-F, ppsA-D-R, ptsG-U-F, ptsG-U-R, ptsG-D-F, ptsG-D-R, crr-U-F, crr-U-R, crr-D-F, crr-D-R, adhE-U-F, adhE-U-R, adhE-D-F, adhE-D-R, ldhA-U-F, ldhA-U-R, ldhA-D-F, ldhA-D-R, zwf-U-F, zwf-U-R, zwf-D-F, and zwf-D-R. The sequences of the poxB-U-F, poxB-U-R, poxB-D-F, poxB-D-R, ppsA-U-F, ppsA-U-R, ppsA-D-F, ppsA-D-R, ptsG-U-F, ptsG-U-R, ptsG-D-F, ptsG-D-R, crr-U-F, crr-U-R, crr-D-F, crr-D-R, adhE-U-F, adhE-U-R, adhE-D-F, adhE-D-R, ldhA-U-F, ldhA-U-R, ldhA-D-F, ldhA-D-R, zwf-U-F, zwf-U-R, and zwf-D-F, zwf-D-R are shown in Table 8 below.

TABLE 8
primers used to construct the upstream and
downstream sequences of the
homology arms of knockout genes

| Primer | Sequence |
|---|---|
| poxB-U-F | GCGGCCCGGCTCCGTATA |
| poxB-U-R | CTCCTGAATGTGATAACGGTAACAAGTTTA |
| poxB-D-F | AAAGGGTGGCATTTCCCGTCA |
| poxB-D-R | AATTCCCATGCTTCTTTCAGGTATTCA |
| ppsA-U-F | AATCTGATCCTTCACTGCCCGTG |
| ppsA-U-R | GACAAAACGCCGCCGGGTATTTATTCGAACAATCCTTTT |
| | GTGATAAATGAACG |
| ppsA-D-F | TCATTTATCACAAAAGGATTGTTCGAATAAATACCCGGC |
| | GGCGTTT |
| ppsA-D-R | TTAACCGCCCGTGCGCTG |
| ptsG-U-F | ATCGGTTACTGGTGGAAACTGACTC |
| ptsG-U-R | CTTAGTCTCCCCAACGTCTTACAGAAATTGAGAGTGCTC |
| | CTGAGTATGGG |
| ptsG-D-F | CCATACTCAGGAGCACTCTCAATTTCTGTAAGACGTTGG |
| | GGAGACTAAGG |
| ptsG-D-R | GTGGATGGGACAGTCAGTAAAGGG |
| crr-U-F | AATTGAAATCGGCGTAATGGTG |
| crr-U-R | GCGCCATTTTTCACTGCGGCAAGAAGATCTTCTCCTAAG |
| | CAGTAAATTGGG |
| crr-D-F | CCAATTTACTGCTTAGGAGAAGATCTTCTTGCCGCAGTG |
| | AAAAATG |
| crr-D-R | ATAGCGGAATTTATGTCAAACCTGA |
| adhE-U-F | AAAATCAAAAAAGGTCTGAATCACG |
| adhE-U-R | TTATATTGCCAGACAGCGCTACTGAAATGCTCTCCTGAT |
| | AATGTTAAACTTTTT |
| adhE-D-F | AGTTTAACATTATCAGGAGAGCATTTCAGTAGCGCTGTC |
| | TGGCAATAT |
| adhE-D-R | ATTAAAAACCATCTGTTTTTGTGGC |
| ldhA-U-F | CAAGCAGAATCAAGTTCTACCATGC |
| ldhA-U-R | CCTGGGTTGCAGGGGAGCGGCAAGAAAGACTTTCTCCAG |
| | TGATGTTGAATCAC |
| ldhA-D-F | ATTCAACATCACTGGAGAAAGTCTTTCTTGCCGCTCCCC |
| | TGCA |
| ldhA-D-R | TGTCTGTTTCGCGGTCGCC |
| zwf-U-F | AGAAACGATTCACCGTCGGTTC |
| zwf-U-R | ATAAAGGATAAGCGCAGATA |
| | GTCATTCTCCTTAAGTTAACTAACCCGG |
| zwf-D-F | GTTAACTTAAGGAGAATGAC |
| | TATCTGCGCTTATCCTTTATGGTTATTT |
| zwf-D-R | TCTGGATAGTGTTCATAAGGCTGGTG |

(5) Amplifying the upstream and downstream DNA sequences of the target gene to be knocked out using their respective primers, and purifying the PCR products according to the following protocol:

Reaction one:
a) upstream 1 uL
b) downstream 1 uL
c) ddH₂O 23 uL
d) PCR enzyme 25 uL
e) Reaction condition: 9 cycles
f) No purification

Reaction two:
a) 8 uL reactant
b) upstream forward primer 1.5 uL
c) downstream reverse primer 1.5 uL
d) ddH₂O 14 uL
e) PCR enzyme 25 uL
f) Purification of PCR products
(6) Transforming the pre-constructed pCas9 plasmid into BL21 strain, inoculating a single colony and culturing overnight at 37° C. Inoculating 1% of the overnight pre-culture and culturing for 1 hour on the next day; inducing promoter expression by adding 10 mM L-arabinose; continue culturing until the OD₆₀₀ reaches approximately 0.6, and then transferring the culture into a tube and incubating on ice for about 15 minutes; harvesting the cells and washing twice with ddH₂0; finally, washing twice with 10% glycerol; resuspending the cells with 400 uL of 10% glycerol and aliquoting 50 uL into a 1.5 mL tube;
(7) Electroporation: 2 uL of donor DNA was added to competent cells, and then 5 uL of pTarget was added and incubated on ice for about 15 minutes. Electroporating the mixture of plasmid and linear DNA, and immediately adding 1 mL of LB/SOB medium. The sample was incubated at 30° C., 220 rpm for 2 hours then plated onto an agar plate containing kanamycin and spectinomycin;
(8) Loss of pTarget plasmid: inoculating the strain containing pCas9 and pTarget, and adding only an equal amount of 0.5 mM IPTG and kanamycin and culturing at 30° C., 220 rpm for 8 to 16 hours. Next, streaking the colonies on a LB agar plates containing only kanamycin and incubating at 30° C. Then, picking colonies and transferring them onto a new agar plate containing both kanamycin and spectinomycin to test for sensitivity to streptomycin, and incubating overnight at 30° C.;
(9) Repeating the process to knock out the remaining 7 genes;
(10) Finally, the pCas9 plasmid was lost by culturing the bacterial strain at 37° C. for approximately 20 hours. The kanamycin sensitivity test was performed in subsequent experiments.

The strains described in this study are listed in Table 9 below.

TABLE 9
strains used in this study

| Strains | Description | Reference |
|---|---|---|
| E. coli BL21 | Expression Host, F⁻ ompT gal dcm hsdS_{B} (r⁻_{B} m⁻_{B}) λ(DE3). | Stratagene |
| E. coli DH5α | Cloning Host, fhuA2 (argF-lacZ) U169 phoA glnV44 ϕ80 | Stratagene |
| | Δ(lacZ)M15 gyrA96 recAl relA1 endA1 thi-1 hsdR17 | |
| N-1 | E. coli BL21, pRSF-NI; pA-ESKKD | this study |
| T-1 | E. coli BL21, pRSF-TI; pA-ESKKD | this study |
| O-1 | E. coli BL21, pRSF-OI; pA-ESKKD | this study |
| N-2 | N-1; pRSF-INI; pA-ESKKD | this study |
| L1 . . . L4 | pRSF-INI (Linker arrays); pA-ESKKD | this study |
| R1 . . . R13 | pRSF-INI (RBS library); pA-ESKKD | this study |
| EL-1 | pRSF-INI (RBS-10); pA-ESKKD | this study |
| EL-2 | EL-1; ΔpoxB; pRSF-INI (RBS-10); pA-ESKKD | this study |
| EL-3 | EL-2; ΔpoxB-ΔppsA; pRSF-INI (RBS-10); pA-ESKKD | this study |
| EL-4 | EL-3; ΔpoxB-ΔppsA-ΔptsG-crr; pRSF-NII (RBS-10); pA-ESKKD | this study |
| EL-5 | EL-4; ΔpoxB-ΔppsA-ΔptsG-crr; pRSF-INI (RBS-10); pA-ESKKD; | this study |
| | pET-GG | |
| EL-6 | EL-5; ΔpoxB-ΔppsA-ΔptsG-crr-ΔadhE; pRSF-INI (RBS-10); pA- | this study |
| | ESKKD; pET-GG | |
| EL-7 | EL-6; ΔpoxB-ΔppsA-ΔptsG-crr-ΔadhE-ΔldhA; pRSF-INI (RBS- | this study |
| | 10); pA-ESKKD; pET-MMGG | |
| EL-8 | EL-7; ΔpoxB-ΔppsA-ΔptsG-crr-ΔadhE-ΔldhA-Δzwf; pRSF-INI | this study |
| | (RBS-10); pA-ESKKD; pET-MMGG | |
| EL-10 | E. coli BL21, pRSF-IN*I (RBS-10); pA-ESKKD | this study |
| EL-11 | EL-8; ΔpoxB-ΔppsA-ΔptsG-crr-ΔadhE-ΔldhA-Δzwf; pRSF-IN*I | this study |
| | (RBS-10); pA-ESKKD; pET-MMGG | |
| β-EL-1 | EL-12; ΔpoxB-ΔppsA-ΔptsG-crr-ΔadhE-ΔldhA-Δzwf; pRSF- | this study |
| | IN*IDG (RBS-10); pA-ESKKD; pET-MMGG | |
| β-EL-2 | EL-12; ΔpoxB-ΔppsA-ΔptsG-crr-ΔadhE-ΔldhA-Δzwf; pRSF- | this study |
| | IN*IA (RBS-10); pA-ESKKD; pET-MMGG | |
| β-EL-3 | β-EL-1; ΔpoxB-ΔppsA-ΔptsG-crr-ΔadhE-ΔldhA-Δzwf; pRSF- | this study |
| | IN*IDG (RBS-10); pA-ESKKD; pET-MMMGGT | |
| β-EL-4 | β-EL-1; ΔpoxB-ΔppsA-ΔptsG-crr-ΔadhE-ΔldhA-Δzwf; pRSF- | this study |
| | IN*IA (RBS-10); pA-ESKKD; pET-MMMGGT | |

### Example 3

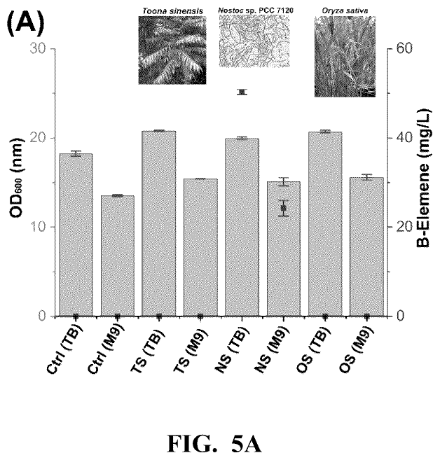

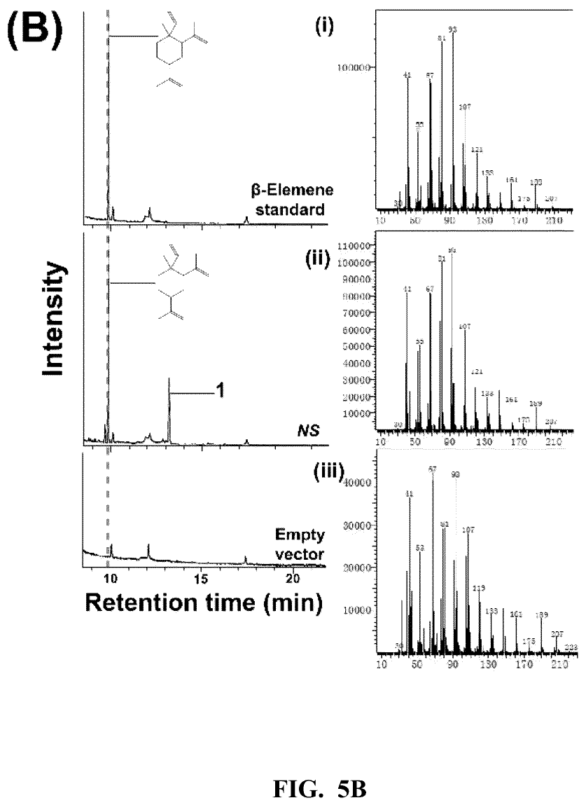

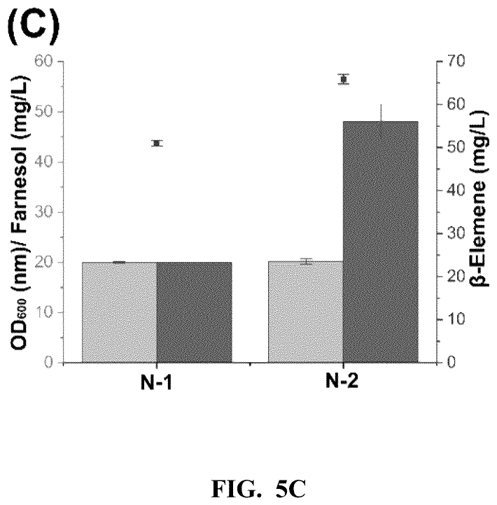

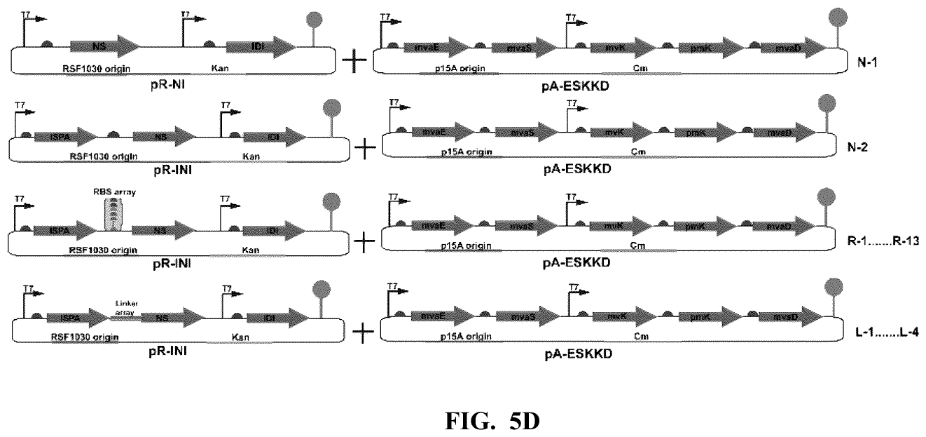

In this example, the synthetase for the de novo biosynthesis of β-elemene was screened, and the steps are as follows:
(1) Co-transforming the codon-optimized synthetic β-elemene synthase gene TS from Toona sinensis, the codon-optimized synthetic β-elemene synthase gene OS from rice, and the codon-optimized synthetic β-elemene synthase gene NS from Nostoc sp. PCC 7120 with the previously constructed plasmid pA-ESKKD that expresses the heterologous MVA pathway, into Escherichia coli BL21 (DE3), yielding strains Ctrl, TS, OS, and NS;
(2) De novo biosynthesis of β-elemene takes place in fermentation medium. The fermentation medium was composed of 7 g/L glucose, 15 g/L yeast extract, 2 g/L KH₂PO₄, 12 g/L K₂HPO₄, 5 g/L tryptone, 1.1 g/L MgSO₄7H₂0, 0.5 g/L citric acid, 4 g/L glycerol. Overnight seed cultures of the above 4 strains were pre-cultured in LB medium at 30° C. at 220 rpm. Inoculating the overnight seeds in 50 ml of fermentation medium to an initial OD₆₀₀ of 0.1 and incubating at 220 rpm at 37° C. for 4 h;
(3) After that, adding 0.5 mM IPTG as an inducer and 20% n-dodecane as an organic layer to capture the product, and incubating at 30° C. and 220 rpm for 72 hours.
(4) Collecting a sample of n-dodecane, diluting it in ethyl acetate according to an appropriate ratio, and using GC-MS to detect and analyze β-elemene and germacrene A. The result is shown in FIGS. 5A-5D. β-elemene was not detected in the two plant-derived terpene synthase (TS and OS) cultures, while NS produced 24.23 mg/L and 50.31 mg/L of β-elemene in M9 and TB mediums, respectively (FIGS. 5A and 5B).

The above results indicate that the optimized Nostoc synthase (NS) produces 1-elemene more efficiently than other terpene synthases. NS was chosen for further research, and the strains expressing pRSF-NI and pA-ESKKD were renamed as N-1.

### Example 4

This example increases the production of β-elemene by overexpressing the ispA enzyme, and the steps are as follows:
(1) The pRSF-INI plasmid carrying the overexpressed Escherichia coli gene ispA and the plasmid pA-ESKKD were co-transformed into Escherichia coli BL21 (DE3) to construct strain N-2;
(2) An overnight seed culture of strain N-2 was pre-cultured in LB medium at 30° C. at 220 rpm. Inoculating the overnight seeds in 50 ml of fermentation medium to an initial OD₆00 of 0.1 and incubating at 220 rpm at 37° C. for 4 h.
(3) After that, adding 0.5 mM IPTG as an inducer and 20% n-dodecane as an organic layer to capture the product, and incubating at 30° C. and 220 rpm for 72 hours.
(4) Collecting a sample of n-dodecane, diluting it in ethyl acetate according to an appropriate ratio, and using GC-MS to detect and analyze β-elemene. As a result, strain N-2 produced 65.89 mg/L of β-elemene. β-elemene concentration was determined as in assay 1. However, increased accumulation of FPP also resulted in the production of 48.05 mg/L farnesol (FOH) by strain N-2 (FIG. 5C).

### Example 5

Referring to FIG. 5D, it shows the plasmid map encoding the β-elemene biosynthesis genes, in which the genes are assembled using different synthetic RBS and linkers into a plasmid map with RSF replication origin (ORI) and kanamycin resistance gene, and co-expressed with the MVA pathway.

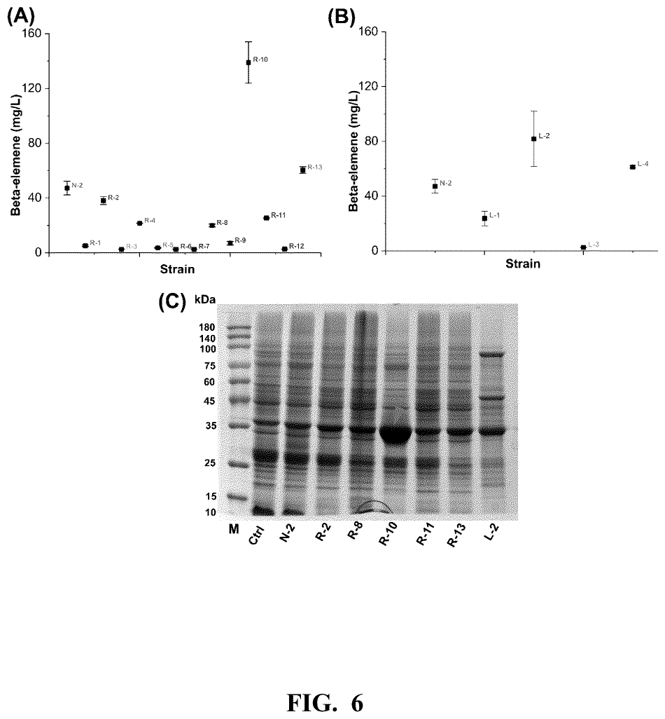

This example is based on RBS engineering to increase the yield of β-elemene, and the steps are as follows:
(1) An electronic RBS library of enzyme NS was generated using the RBS calculator, and these RBS with translation initiation rates (TIR) from 500 arbitrary units (au) to 50,000 au were added to the 5′ end of NS to generate plasmid pRSF-INI113 (FIG. 6A);
(2) Adding RBS to the 5′ end of primer F1 to amplify gene NS with primer F1/R2. The sequences of primers F1 and R2 are shown in Table 1. Farnesyl pyrophosphate (ispA) was amplified from Escherichia coli genomic DNA using primers ispA-F/ispA-R. Cloning these two fragments into NcoI/BamHI digested Prsf-NI to generate plasmids pR1, pR2, pR3, pR4, pR5, pR6, pR7, pR8, pR9, pR10, pR11, pR12, pR13;
(3) Plasmids pR1, pR2, pR3, pR4, pR5, pR6, pR7, pR8, pR9, pR10, pR11, pR12, pR13 and plasmid pA-ESKKD were transformed into Escherichia coli respectively, and 13 strains were constructed: R1, R2, R3, R4, R5, R6, R7, R8, R9, R10, R11, R12 and R13, their RBS sequences and predicted TIRs are shown in Table 10.

TABLE 10
RBS sequence and translation initiation
rate (TIR) for gene NS

| RBS sequence | TIR |
|---|---|
| GACCAACCTCAACTAGGATTAGCCT | 520.23 |
| GATACCTAATCTCCTTAAGTTTCGTCTTCC | 966.71 |
| CGCGTAAACCCACCGAGATCCGAGACCTTTG | 2556.52 |
| ACAATCCATACGAAAATTAAGAGACTGATTA | 3649.28 |
| CTTCCCTTAAAGGGTCTGCAAATAAATCAGAGGAAATT | 4568.74 |
| TACCTAGTCTACCCCGAAATCTGTTGGAGTTTTTT | 6872.74 |
| GCTTATCTGAAAGTTTTAGTCGAGGATTTTT | 9357.01 |
| AAGTAAACTAACTTTAAGGAGTCCTT | 11711.8 |
| CGAAGCCCGTCGAACCAAGACTATCCAAATTTAAGTAG | 13459.92 |
| GAATTTT | |
| TCGTTGCAACCGACTTCAAGGAGACTTTTAT | 18865.61 |
| GACCCTTGTTAAGCCCCGAAAAGGAGGAGTAAAT | 27050.12 |
| TAGTCGAAATCTATAAGGAGAATTAT | 42070.81 |
| CCATCACTTGGACTTCCGCGTATATAAGGAGGTTTTT | 45297.49 |

(4) The overnight seed cultures of 13 strains R1 to R13 were pre-cultured in LB medium at 30° C. at 220 rpm. Inoculating the overnight seeds in 50 ml of fermentation medium to an initial OD₆₀₀ of 0.1 and incubating at 220 rpm at 37° C. for 4 h.
(5) After that, adding 0.5 mM IPTG as an inducer and 20% n-dodecane as an organic layer to capture the product, and incubating at 30° C. and 220 rpm for 72 hours. The fermentation results showed that strain R10 was the best strain, producing 151.25 mg/L of β-elemene in a 50 mL shake flask, which was 3.27 times higher than that of strain N-2.

### Example 6

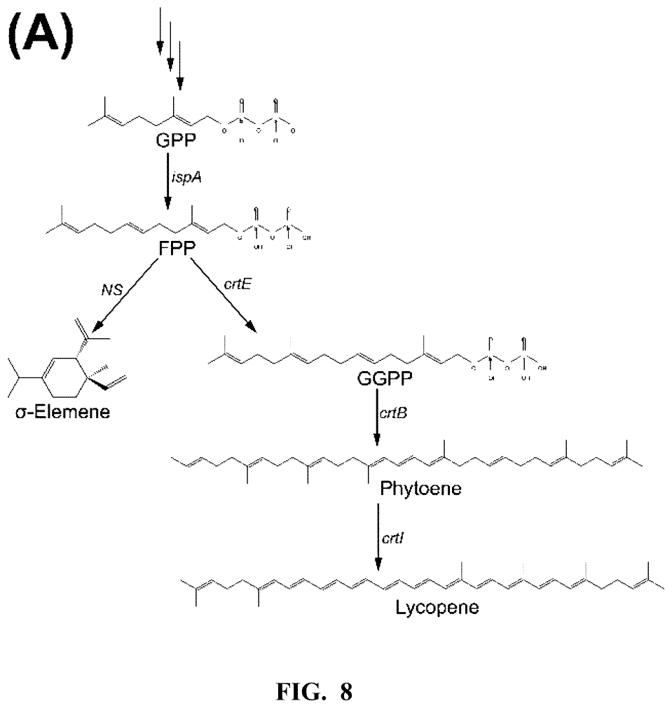

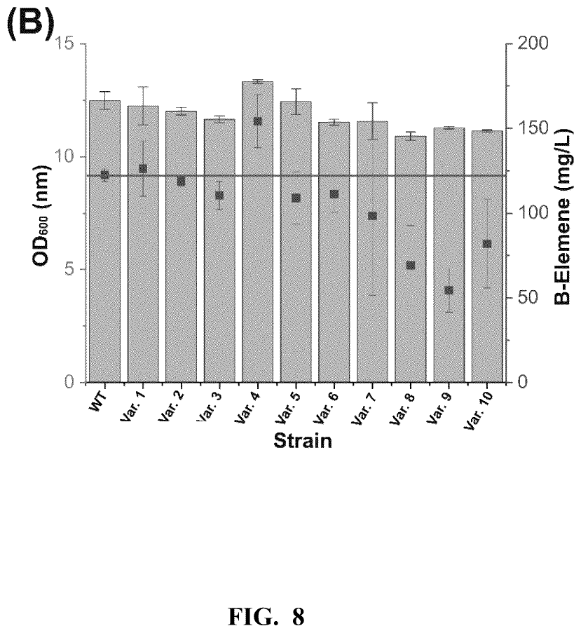

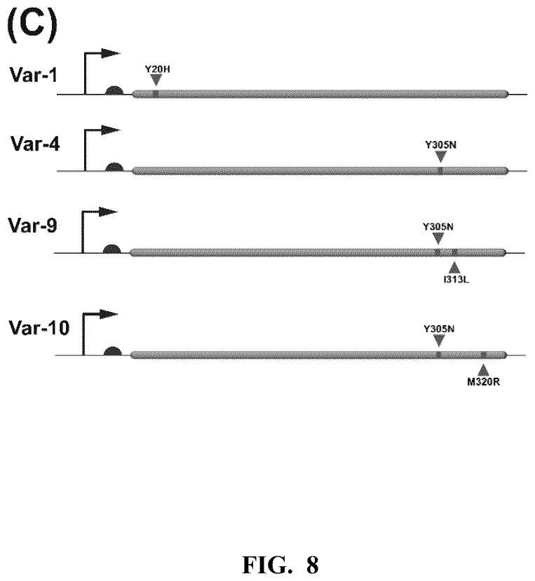

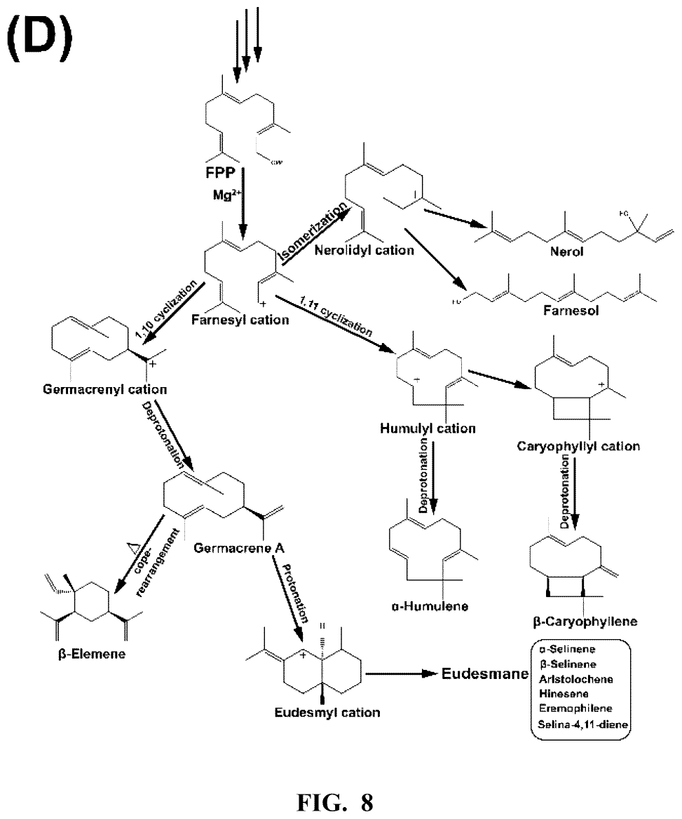

In this example, the production of β-elemene is improved by protein fusion technology, and the steps are as follows:
(1) Referring to FIG. 6B, fusing two enzymes ispA and NS using four different linkers to fuse the N-terminus of type I non-plant synthase NS with the C-terminus of an endogenous ispA, the four different linkers include a short chain linker, a medium chain linker, a long chain linker, and a flexible chain linker, and the short chain is GGGS, the medium chain is (GGGS)2, the long chain is (GGGS)3, and the flexible chain is GGGGS. Each linker sequence is shown in Table 11; the stop codon of ispA was removed during this process. The resulting fusion plasmids were pL1, pL2, pL3 and pL4, respectively. These four fusion plasmids were transformed into Escherichia coli with plasmid pA-ESKKD respectively to construct strains L1, L2, L3, and L4. GC-MS analysis results showed that the best fusion strain L-2 produced 85.89 mg/L, and compared with R10, the production of β-elemene by the fusion enzyme was reduced (FIG. 8B). β-elemene concentration was determined as in assay 1;
(2) To confirm the correlation between protein levels and the total amount of β-elemene produced, the present invention selected five representatives (R2, R8, R10, R11, and R13) from the panel RBS series in Example 5 and one representative from the fusion expression strain L2 for SDS-PAGE analysis. As expected, R10 had a higher NS protein band with a size of 37.51 kDa compared with other strains (FIG. 6C). R10 was selected for subsequent experiments and was renamed EL-1.

TABLE 11
linker sequences for short chain, medium chain,
long chain, and flexible
chain for fusion of ispA and NS enzymes

| Linker name | Nucleic acid sequence |
|---|---|
| Short chain linker | GGTGGTGGTTCT |
| Medium chain linker | GGTGGTGGTTCTGGTGGTGGTTCT |
| Long chain linker | GGTGGTGGTTCTGGTGGTGGTTCTGGT |
| | GGTGGTTCT |
| Flexible chain linker | GGTGGTGGTGGTTCT |

### Example 7

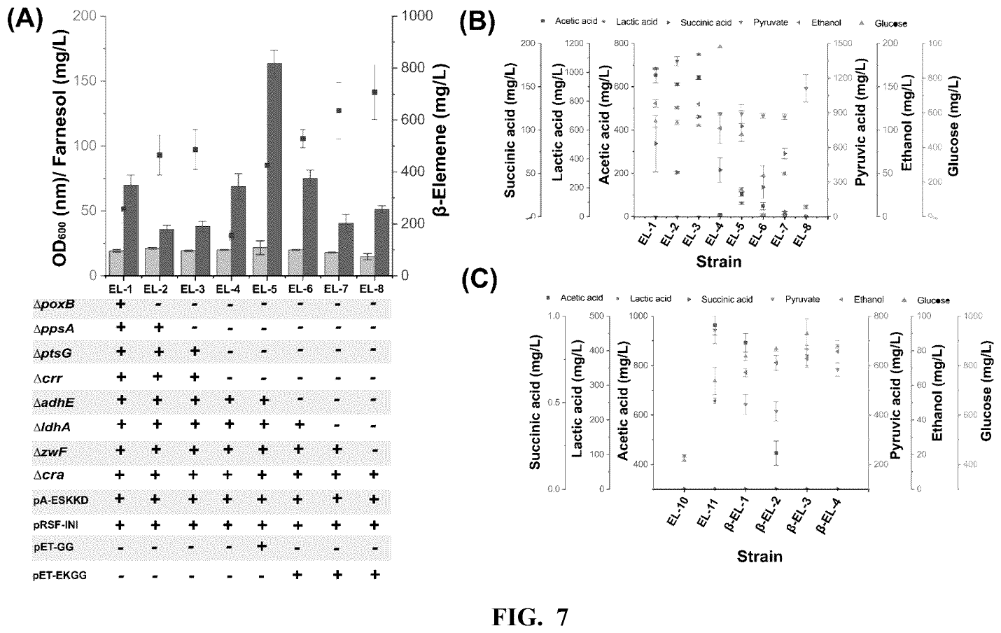

In this example, the precursor accumulation is improved by rewriting the central carbon metabolism pathway, and the steps are as follows:
(1) Co-transforming the pR10 plasmid and pA-ESKKD plasmid into defective bacteria ΔpoxB, ΔpoxBΔppsA, ΔpoxBΔappsAΔptsGΔcrr to obtain strains EL-2, EL-3, EL-4; the pET-GG, pR10 plasmid and pA-ESKKD plasmid expressing glk and galP genes were transformed into strain ΔpoxBΔppsAΔptsGΔcrr to obtain strain EL-5; The pET-EKGG plasmid, pR10 plasmid and pA-ESKKD plasmid expressing mvaE, mvk, glk and galP genes were transformed into strains ΔpoxBΔppsAΔptsGΔcrrΔadhE, ΔpoxBΔppsAΔptsGΔcrrΔadhEΔldhA, ΔpoxBΔppsAΔptsGΔcrrΔadhEΔldhAΔzwf and recombinant bacteria EL-6, EL-7, EL-8;
(2) Three colonies each of strains EL-1, EL-2, EL-3, EL-4, EL-5, EL-6, EL-7 and EL-8 were inoculated into LB medium, and overnight seed cultures were grown in pre-incubate at 30° C. at 220 rpm. Inoculating the overnight seeds separately in 50 mL of fermentation medium in 250 mL Erlenmeyer flasks to an initial OD₆₀₀ of 0.1 and incubate at 37° C. at 220 rpm for 4 h;
(3) After that, adding 0.5 mM IPTG as an inducer and 20% n-dodecane as an organic layer to capture the product, and incubating at 30° C. and 220 rpm for 72 hours;
(4) Collecting a sample of n-dodecane, diluting it in ethyl acetate according to an appropriate ratio, and using GC-MS to detect and analyze β-elemene. EL-1 and EL-2 finally produced 257.56 mg/L and 465.32 mg/L of β-elemene, respectively (FIG. 7A), among which, strain EL-2 did not show a significant reduction in acetate accumulation (FIG. 7B).

Strain EL-3 produced 486.43 mg/L of β-elemene, 68.79 mg/L of FOH, and the L-lactate level in strain EL-3 was as high as 1124.05 mg/L, and the inactivation of ppsA resulted in lactic acid and ethyl salt accumulation increased (FIG. 7B).

The β-elemene production of the PTS-deficient strain EL-4 was reduced, and the FOH production was increased to 68.79 mg/L, and the cell growth was not significantly affected (FIG. 7A). Pyruvate was excessively accumulated in PTS-deficient strains (FIG. 7B), so it was speculated that PTS deficiency resulted in insufficient intracellular glucose supply and reduced production of β-elemene.

The strain EL-5 overexpressing the genes galP and glk produced 425.39 mg/L of 3-elemene, while the production of FOH increased to 81.84 mg/L. OD₆₀₀ was restored in the EL-5 strain.

Strain EL-6 reduced carbon flux to ethanol and increased precursor accumulation yielded 528.83 mg/L of 3-elemene and 37.66 mg/L of FOH (FIG. 7A).

To further increase MVA pathway flux, ldhA was knocked out and mvaE and mvk were overexpressed in strain EL-6 to form strain EL-7, which produced 636.40 mg/L of 1-elemene (FIG. 7A). Strain EL-8 with the deletion of the zwf gene produced 707.18 mg/L 1-elemene and 25.51 mg/L FOH (FIG. 7A). The increased production of β-elemene suggests that zwf loss directs carbon metabolic flux towards isoprenoid production.

### Example 8

In this example, a high-activity NS enzyme is obtained through high-throughput screening of directed evolution enzyme NS, and the steps are as follows:

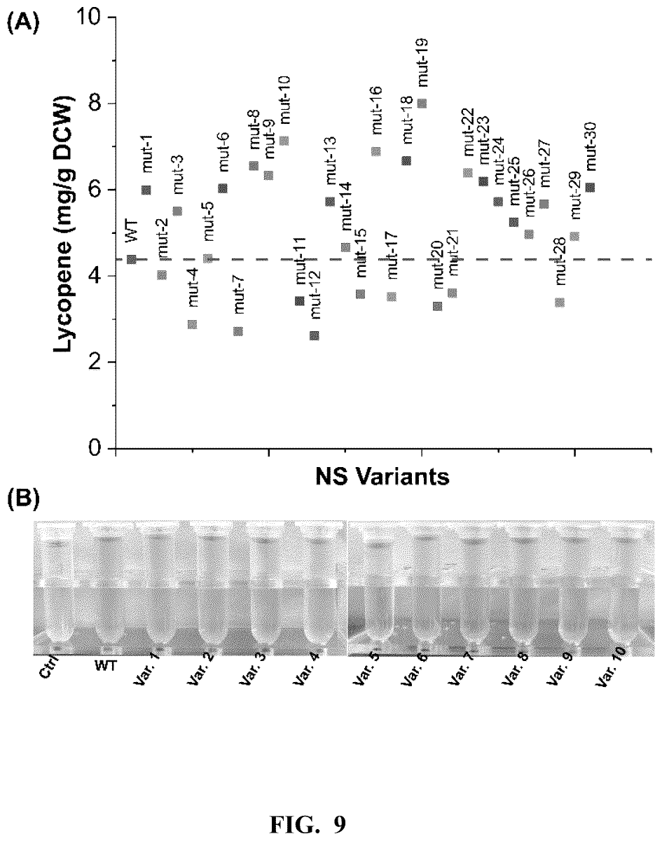

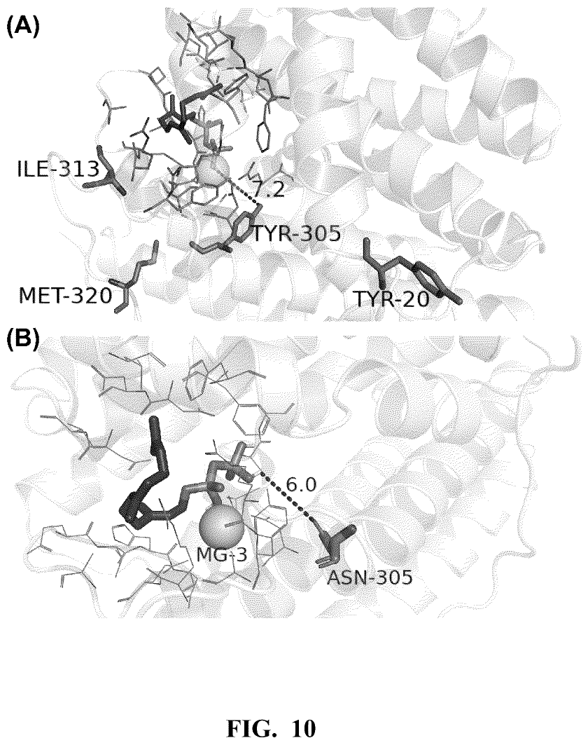

The universal substrate FPP serves as a precursor for the downstream synthesis of sesquiterpenes and tetraterpenes (FIG. 8A). Since β-elemene and lycopene share this common precursor FPP, it was hypothesized that improved NS would pull more FPP towards β-elemene production, thereby reducing lycopene coloration. Therefore, lycopene staining was used as a high-throughput screening method to enhance NS activity.
(1) Error-prone PCR mutants were generated using the Diverse PCR Random Mutagenesis Kit (Vazyme). The system for error-prone PCR reactions was prepared according to the manufacturer's instructions. The mutants were cloned into the plasmid pRSFDuet-IDI to generate a gene library of the mutant pRSF-N*I, and the gene library plasmid pRSF-N*I was transformed into E. coli overexpressing the plasmid pA-ESKKD of MVA and the lycopene pathway;
(2) Picking a single colony from the mutant pool into the fermentation medium in a deep-well plate using a sterile pipette tip, and incubating at 30° C. and 220 rpm for 72 h. Lycopene was extracted, and colonies in 48-well plates were screened by identifying the coloration of cells in acetone, and strains with light lycopene color were selected for subsequent rounds of selection. At the same time, the production of lycopene was measured to verify the results (FIG. 9A shows the production of lycopene in both the wild-type strain and 10 screened mutants within 24 hours; FIG. 9B shows the color of the extracted lycopene). Lycopene assays are shown below;
(3) Then, the colonies in 48-well plates were screened by identifying staining of cells in acetone. Ultimately 10 variants were selected from 3 rounds of screening. The point mutation from Y305 to N305 resulted in the highest production of β-elemene (FIG. 8B-8C), that is, the amide amino acid mutation might have improved the binding ability of FPP at the NS active site;
(4) To analyze the reason for the higher activity, molecular docking was performed to predict the interaction between Y305N (receptor) and FPP (ligand), and the best docking model was visualized using the PyMOL viewer. Using the Swiss model to build an online tool network (https://swissmodel.expasy.org/) to construct a homology model of NS. The model line is based on the structure of the selinadiene synthase apo and complex diphosphate (PDB: 40KM) with 25.08% sequence identity and 98% sequence coverage (3-322 of 322 residues) with NS; molecular docking was performed using PyRx (https://pyrx.sourceforge.io), the 3D structure of FPP was downloaded from PubChem.org, while visualization and labeling was done using PyMoL software (https://pymol.org). The results show that the hydrogen bonds between residues Y305 and N305 and the ligand are 7.2 Å and 6.0 Å, respectively (FIGS. 10A-10B), indicating that N305 is close to FPP. In addition, molecular docking calculations showed that the binding affinities between the receptor and ligand of Y305 and N305 were 7.2 kcal/mol and 6.8 kcal/mol, respectively. This indicated that the enzyme N305 could combine with the receptor FPP unhindered, and the enzymatic activity of NS was enhanced; (5) GC-MS product profile analysis of selected NS mutants showed that 80% of the variants still produced β-elemene as the major product. However, var-9 and var-10 with double mutations at Y305N/I313L and Y305N/M320R resulted in the production of a mixture of sesquiterpenes. The mixture had a significant match to compounds in the National Institute of Standards and Technology (NIST) database, which was also identified using the compounds' mass spectra and retention indices. This suggests that a variety of sesquiterpenes can be produced from the universal substrate FPP by simple cyclization and rearrangement reactions of sesquiterpene synthases (FIG. 8D);
(6) The overnight seed cultures of the positive variants Y20H and Y305N as well as the wild-type strain were pre-cultured in LB medium at 220 rpm at 30° C. Inoculating the overnight seeds in 50 ml of fermentation medium to an initial OD₆₀₀ of 0.1 and incubate at 220 rpm at 37° C. for 4 h;
(7) After that, adding 0.5 mM IPTG as an inducer and 20% n-dodecane as an organic layer to capture the product, and incubating at 30° C. and 220 rpm for 72 hours;
(8) Collecting a sample of n-dodecane, diluting it in ethyl acetate according to an appropriate ratio, and using GC-MS to detect and analyze β-elemene. The positive variants Y20H and Y305N produced 126.16 mg/L and 154.14 mg/L of β-elemene, respectively. The β-elemene production of these mutants was increased by 2.52-fold and 3.07-fold compared with the wild type (FIG. 8B).

In combination with the embodiments of Examples 3-8, in order to increase the productivity, the NS in the plasmid pRSF-INI containing RBS-10 was replaced with NS (Y305N), that is, the plasmid pRSF-IN*I. Plasmid pRSF-IN*I was co-transformed with pET-EKGG plasmid and pA-ESKKD plasmid into WT strain and knockout bacteria ΔpoxBΔppsAΔptsGΔcrrΔadhEΔldhAΔzwf, to obtain strains EL-10 and EL-11.

The overnight seed cultures of the above strains were pre-cultured in LB medium at 30° C. at 220 rpm. Inoculating the overnight seeds in 50 ml of fermentation medium to an initial OD₆₀₀ of 0.1, and incubating at 220 rpm at 37° C. for 4 h.

After that, adding 0.5 mM IPTG as an inducer and 20% n-dodecane as an organic layer to capture the product, and incubating at 30° C. and 220 rpm for 72 hours.

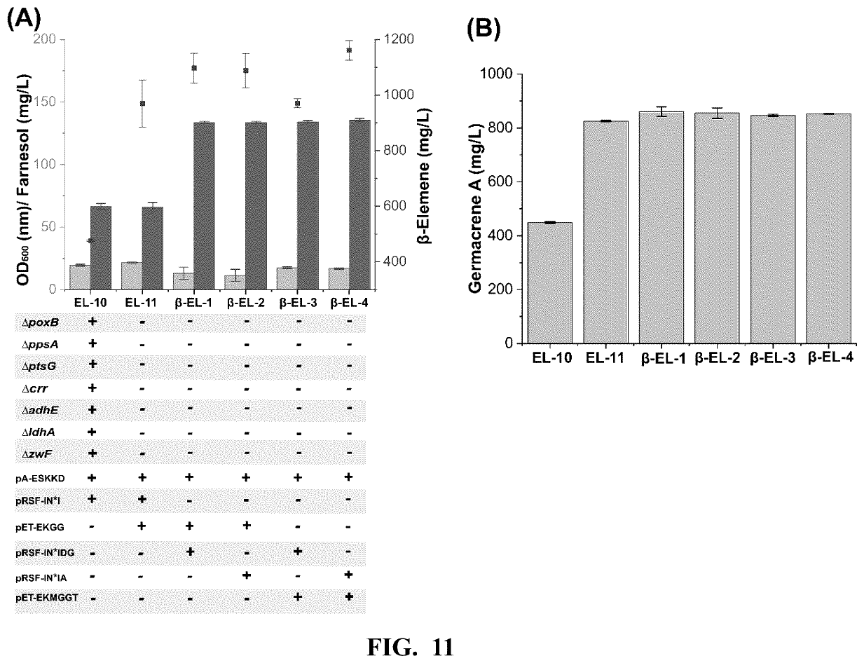

Collecting a sample of n-dodecane, diluting it in ethyl acetate according to an appropriate ratio, and using GC-MS to detect and analyze β-elemene. Strain EL-10 produced 480.55 mg/L of β-elemene and 66.5 mg/L of FOH. Compared with EL-1, the production of β-elemene increased by 1.87 times. The performance of NS(Y305N) for β-elemene production in strain EL-8 was evaluated. The EL-11 strain carrying the plasmid pRSF IN*I (RBS-10) produced 970.15 mg/L of β-elemene, a 37% increase compared to that of EL-8 (FIG. 11A).

### Example 9

In this example, the yield of β-elemene is improved by adjusting the metabolism to recycle the accumulated pyruvate. The steps are as follows:
(1) Since pyruvate is still the major “sink” for carbon flux in strain EL-8, in order to drive pyruvate into the downstream MEP and MVA pathways of β-elemene biosynthesis, 1-deoxyxylone-5-phosphate synthase (dxs), 1-hydroxy-2-methyl-2-(E)-Butenyl 4-diphosphate synthase (ispG) and pforA were cloned into plasmid pRSF-IN*I, resulting in plasmids pRSF-IN*IDG and pRSF-IN*IA, respectively. The resulting plasmids were expressed in the knockout strain ΔpoxBΔppsAΔptsGΔcrrΔadhEΔldhΔzwf together with the pA-ESKKD plasmid and pET-EKGG plasmid to produce strains β-EL-1 and β-EL-2;
(2) The overnight seed cultures of strains β-EL-1 and β-EL-2 were pre-cultured in LB medium at 30° C. at 220 rpm. Inoculating the overnight seeds in 50 ml of fermentation medium to an initial OD₆₀₀ of 0.1, and incubating at 220 rpm at 37° C. for 4 h;
(3) After that, adding 0.5 mM IPTG as an inducer and 20% n-dodecane as an organic layer to capture the product, and incubating at 30° C. and 220 rpm for 72 hours;
(4) Collecting a sample of n-dodecane, diluting it in ethyl acetate according to an appropriate ratio, and using GC-MS to detect and analyze β-elemene. In β-EL-1 and β-EL-2, the production increased to 1096.55 mg/L and 1088.63 mg/L, respectively (FIG. 11A). Thus, “flushing” of the pyruvate pool by dxs, ispG and pforA directs more carbon flux towards β-elemene synthesis.

### Example 10

This example promotes the production of β-elemene by regulating the efflux pump, and the steps are as follows:
(1) Most monoterpenes and sesquiterpenes are harmful to cells, and efflux engineering is used to increase cellular efflux and tolerance to the product. Therefore, the outer membrane protein tolC and the inner membrane lipopolysaccharide msbA of the efflux pump were overexpressed based on strains β-EL-1 and β-EL-2. Transferring plasmids of pET-EKMGGT and pA-ESKKD overexpressing an outer membrane protein tolC and an inner membrane lipopolysaccharide msbA of the efflux pump into the defective strain ΔpoxBΔppsAΔptsGΔcrrΔadhEΔldhAΔzwf along with plasmids of pRSF-IN*IDG and pRSF-IN*IA, respectively, to obtain strains β-EL-3 and β-EL-4;
(2) Pre-culturing the overnight seed cultures of the above 2 strains in LB medium at 30° C. at 220 rpm. Inoculating the overnight seeds in 50 ml of fermentation medium to an initial OD₆₀₀ of 0.1, and incubating at 220 rpm at 37° C. for 4 h;
(3) After that, adding 0.5 mM IPTG as an inducer and 20% n-dodecane as an organic layer to capture the product, and incubating at 30° C. and 220 rpm for 72 hours;
(4) Collecting a sample of n-dodecane, diluting it in ethyl acetate according to an appropriate ratio. Detection analysis was performed using GC-MS. β-EL-3 and β-EL-4 produced 970.68 mg/L and 1161.09 mg/L of β-elemene, respectively. Overexpression of these efflux genes also improved cell growth (FIG. 11A), suggesting that efflux engineering attenuates cytotoxicity. In the GC-MS column, germacrene A was not completely converted to β-elemene, and the residual amount was about 852.36 mg/L (FIG. 11B), which indicated that the total germacrene A produced in the shake flask was about 2013.45 mg/L.

Referring to the pathway optimization approach in Example 7 to treat strains EL-10, EL-11, β-EL-1, β-EL-2, β-EL-3, β-EL-4, and record the effect of pathway optimization on organic acid accumulation in different strains. The results are shown in FIG. 7C.

### Example 11

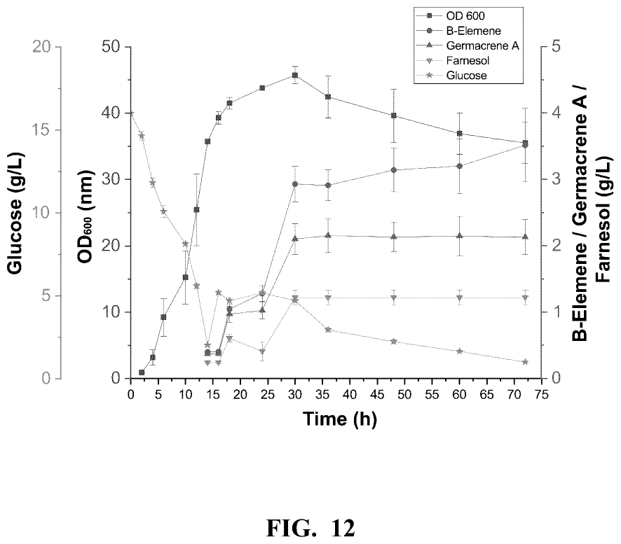

The present invention selects recombinant bacterium β-EL-4 as research object according to the embodiment of Examples 3-10, and tests its ability to produce β-elemene under fed-batch fermentation:
(1) A 4-L bioreactor was used for fed-batch fermentation, and 2.5-L fermentation medium was added to produce β-elemene;
(2) The β-EL-4 strain was incubated as a seed strain and inoculated with an initial OD₆₀₀ of 0.1. Glucose was initially added to the medium at 16 g/L, while an exponential-to-do-stat feeding strategy was used to maintain a constant supply of glucose. The fermentation medium contains: K₂HPO₄ 3 g/L; KH₂PO₄ 2 g/L; (NH₄)25O₄ 5 g/L; yeast powder 7 g/L; tryptone 5 g/L; MgSO₄·7H₂O 1 g/L; anhydrous CaCl₂ 55 mg/L; anhydrous MnSO₄ 25 mg/L; FeSO₄ 167 mg/L; thiamine 0.25 mM; glucose 16 g/L and glycerol 4 g/L;
(3) Dissolved oxygen (DO) was kept constant at 10% by adjusting the stirring rate in the range of 300 to 800 rpm. The pH was maintained at 7 by titration with ION NH₄OH and 5N H₂SO₄. After 12 hours at 37° C. and upon induction with 0.5 mM IPTG, the temperature was switched to 30° C. After IPTG induction, 20% dodecane was added as the extraction phase, and concentrated glucose (60% w/v) was added to maintain sufficient carbon supply and cell growth;
(4) Samples were taken regularly to detect the OD₆₀₀ of the cells, as well as the concentration of β-elemene, FOH, germacrene A, glucose and various organic acids. The experimental results are shown in FIGS. 12A-12B. In the first 24 hours, the strain rapidly utilized glucose, and β-elemene rapidly accumulated to about 1.29 g/L; then, the cells entered the stationary phase, the growth decreased, and the production of β-elemene continued, reaching nearly 3 g/L; finally, after 72 hours, the concentration of β-elemene gradually increased to 3.52 g/L. After Cope-Claisen rearrangement by GC-MS, the residual germacrene A concentration was 2.13 g/L, while the FOH concentration reached 1.22 g/L (FIG. 12). Acetate, lactate, succinate and pyruvate accumulated to higher concentrations before IPTG induction but were barely detected after fermentation. This indicates that strain β-EL-4 efficiently utilizes them as a carbon source for the production of β-elemene.

The present invention describes a Escherichia coli cell factory that produces high levels of β-elemene and germacrene A. Firstly, β-elemene and germacrene A are synthesized from scratch through the screening of germacrene A synthase and the overexpression of the mevalonate pathway; then, the availability of acetyl-CoA, pyruvate, and glyceraldehyde-3-phosphate in the farnesyl diphosphate pathway is ensured by deleting competing pathways in the central carbon metabolism; next, the present invention uses lycopene color as a high-throughput screening method and obtains an optimized NSY305N through error-prone PCR. Finally, in shake flasks, strain β-EL-4 constructed through key pathway enzymes, efflux engineering, and translation engineering produced 1161.09 mg/L of β-elemene and 852.36 mg/L of germacrene A, which is the highest reported yield at shake flask level. In 4-L fed-batch fermentation, the production of β-elemene and germacrene A reached 3.52 g/L and 2.13 g/L, respectively.

The present invention analyzed the biosynthetic and regulatory mechanisms of sesquiterpenes during the process of constructing a high-yield β-elemene-producing chassis cell. This process is applicable for microbial production of a wider range of chemicals and also indicates the feasibility of central metabolism engineering of recombinant Escherichia coli for cost-effective, large-scale production of acetyl-CoA and pyruvate-derived compounds. The techniques and strategies developed in this study provide a successful example and reference for microbial production of a wider range of terpenoid compounds.

It is worth noting that the foregoing examples are only used for illustration of the technical solutions of the present invention and non-limiting thereto. Though reference is made to preferred examples for detailed illustration of the present invention, a skilled person in the art should understand that the technical solutions provided by the present invention can vary or be substituted by equivalents without departing from the spirit and scope of the technical solutions described herein, which should fall within the scope of the appended claims.

What is claimed is:

1. A method for constructing a recombinant bacterium with high production of β-elemene and germacrene A, comprising:
selecting a synthetic enzyme with high efficiency in de novo biosynthesis of β-elemene;
increasing the production of β-elemene by overexpressing an enzyme ispA;
further increasing the production of β-elemene through RBS engineering or protein fusion technology;
enhancing a precursor accumulation by rewriting a central carbon metabolism pathway;
high-throughput screening of a directed evolution enzyme NS to obtain a highly active NS enzyme;
increasing the production of β-elemene by adjusting metabolism to recycle accumulated pyruvic acid;
adjusting an efflux pump to promote the production of β-elemene; and
constructing the recombinant bacterium with high yield of β-elemene and germacrene A.

2. The method of claim 1, wherein the synthetic enzyme comprises β-elemene synthase genes of TS (NCBI Accession number: AB730585) from Toona sinensis, OS (NCBI Accession number: ABJ16553) from rice, and NS (NCBI Accession number: BAB76384) from Nostoc sp. PCC7120, with sequences identified as SEQ ID NO: 1, SEQ ID NO: 2, and SEQ ID NO: 3, respectively.

3. The method of claim 1, wherein the step of increasing the production of β-elemene by overexpressing an enzyme ispA comprises co-transforming a plasmid pRSF-INI carrying an overexpressed gene ispA of Escherichia coli with a plasmid pA-ESKKD carrying a metabolic pathway for synthesizing β-elemene and germacrene A into Escherichia coli BL21 (DE3), the sequence of the plasmid pA-ESKKD is shown as SEQ ID NO: 4.

4. The method of claim 1, wherein the step of increasing the production of β-elemene through RBS engineering comprises generating an electronic RBS library for the enzyme NS using an RBS calculator, and adding RBS sequences with translation initiation rates (TIR) ranging from 500 to 50,000 arbitrary units to the 5′ end of the NS to create a plasmid pRSF-INI.

5. The method of claim 1, wherein the step of increasing the production of β-elemene through protein fusion technology comprises fusing the enzyme ispA and the enzyme NS using four different linkers to fuse the N-terminus of type I non-plant synthase NS with the C-terminus of an endogenous ispA, the four different linkers comprise a short chain linker, a medium chain linker, a long chain linker, and a flexible chain linker, and the short chain is GGGS, the medium chain is (GGGS)2, the long chain is (GGGS)3, and the flexible chain is GGGGS.

6. The method of claim 1, wherein the step of enhancing a precursor accumulation by rewriting a central carbon metabolism pathway comprises knocking out one or more genes encoding pyruvate dehydrogenase (poxB), phosphoenolpyruvate synthase (ppsA), glucose-specific phosphotransferase system enzyme (ptsG), glucose-specific IIA component of the phosphotransferase system (crr), acetaldehyde dehydrogenase (adhE), D-lactate dehydrogenase (ldhA), and glucose-6-phosphate dehydrogenase (zwf) in the wild-type strain Escherichia coli BL21 (DE3), resulting in the defective strain ΔpoxBΔppsAΔptsGΔcrrΔadhEΔldhAΔzwf; and overexpressing one or more genes encoding galactose permease (galP) and glucose kinase (glk);
wherein the sequence of knocked-out gene poxB is shown as SEQ ID NO: 5;
the sequence of knocked-out gene ppsA is shown as SEQ ID NO: 6;
the sequence of knocked-out gene ptsG is shown as SEQ ID NO: 7;
the sequence of knocked-out gene crr is shown as SEQ ID NO: 8;
the sequence of knocked-out gene adhE is shown as SEQ ID NO: 9;
the sequence of knocked-out gene ldhA is shown as SEQ ID NO: 10;
the sequence of knocked-out gene zwf is shown as SEQ ID NO: 11.

7. The method of claim 1, wherein the step of increasing the production of β-elemene by adjusting metabolism to recycle accumulated pyruvic acid comprises cloning 1-deoxy-D-xylulose-5-phosphate synthase (dxs), 1-hydroxy-2-methyl-2-(E)-butenyl 4-diphosphate synthase (ispG), and pforA into the plasmid pRSF-INI to generate plasmids of pRSF-INIDG and pRSF-IN*IA, respectively, the plasmids are then expressed in the defective strain ΔpoxBΔppsAΔptsGΔcrrΔadhEΔldhAΔzwf along with plasmids of pA-ESKKD and pET-EKGG;
wherein the sequence of gene dxs is shown as SEQ ID NO: 12; the sequence of gene ispG is shown as SEQ ID NO: 13; the sequence of plasmid pRSF-IN*IA is shown as SEQ ID NO: 14.

8. The method of claim 1, wherein the step of adjusting an efflux pump to promote the production of β-elemene comprises transferring plasmids of pET-EKMGGT and pA-ESKKD overexpressing an outer membrane protein tolC and an inner membrane lipopolysaccharide msbA of the efflux pump into the defective strain ΔpoxBΔppsAΔptsGΔcrrΔadhEΔldhAΔzwf along with plasmids of pRSF-IN IDG and pRSF-IN*IA, respectively;
wherein the sequence of plasmid pET-EKMGGT is shown as SEQ ID NO: 15.

9. The method of claim 1, wherein the recombinant bacterium obtained by the method produces 1161.09 mg/L of β-elemene and 852.36 mg/L of germacrene A through fermentation in a shaking flask.

10. The method of claim 1, wherein the recombinant bacterium obtained by the method produces 3.52 g/L of β-elemene and 2.13 g/L of germacrene A in a 4-L fed-batch fermentation.
